(19) United States
(12) Patent Application Publication
(10) Pub. No.: US 2010/0021907 A1
(43) Pub. Date: Jan. 28, 2010

(54) METHOD OF DETECTING A PLURALITY OF NUCLEIC ACIDS

(75) Inventors: Jun OKADA, Tokyo (JP); Nobuhiro Gemma, Yokohama-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: KABUSHIKI KAISHA TOSHIBA, Tokyo (JP)

(21) Appl. No.: 12/483,840

(22) Filed: Jun. 12, 2009

(30) Foreign Application Priority Data
Jul. 3, 2008 (JP) 2008-174927

(51) Int. Cl. C12Q 1/68; C12M 1/34

(52) U.S. Cl. 435/6; 435/287.2

(57) ABSTRACT

The present invention provides a method of detecting a plurality of nucleic acid samples, includes a first step of preparing a nucleic acid sample detection device, a second step of preparing 1^{st} to n^{th} nucleic acid sample discrimination reagents, a third step of adding the 1^{st} to n^{th} nucleic acid sample discrimination reagents to 1^{st} to n^{th} nucleic acid samples respectively, a fourth step of injecting the 1^{st} to n^{th} nucleic acid samples into 1^{st} to n^{th} wells respectively, a fifth step of detecting the presence or absence of a reaction in positive control immobilization regions in the 1^{st} to n^{th} wells, and a sixth step of detecting the presence or absence of a reaction in detection nucleic acid probe immobilization regions in the 1^{st} to n^{th} wells.

### CROSS-REFERENCE TO RELATED APPLICATIONS

This application is based upon and claims the benefit of priority from prior Japanese Patent Application No. 2008-174927, filed Jul. 3, 2008, the entire contents of which are incorporated herein by reference.

### BACKGROUND OF THE INVENTION

1. Field of the Invention

The present invention relates to a method of detecting a nucleic acid sample by a nucleic acid sample detection device having nucleic acid probes immobilized thereon, and in particular, to a method of detecting a plurality of nucleic acid samples at one time.

2. Description of the Related Art

With the development of molecular biology in recent years, many disease genes have been identified, which has made the identification of diseases by genetic diagnosis possible. Tailor-made medicines are now being realized which, on the basis of results on genetic diagnosis, provide optimum treatment to individual patients.

As the effectiveness of genetic diagnosis increases, the number of samples handled in the clinical field increases drastically, so examination arrays and examination methods for examining a lot of nucleic acid samples simultaneously are strongly desired, and some have already been realized (Jpn. Pat. Appln. KOKAI Publication No. 2005-345243).

However, when a lot of nucleic acid samples are simultaneously examined, there arise problems such as mix-up of samples and contamination. Genetic diagnosis is often preclinical diagnosis, and based on the diagnostic results, preventive therapy is conducted frequently, so acquisition of accurate diagnostic results is essential.

### BRIEF SUMMARY OF THE INVENTION

The object of the present invention is to provide a detection method that can prevent false detection by mix-up of samples and contamination among samples and is endowed with high safety and reliability required in the clinical field.

### First Embodiment

The present invention provides, as a first embodiment, a method of detecting a plurality of nucleic acid samples, wherein 1^{st} to n^{th} (n: a natural number of 2 or more) nucleic acid samples are detected on a nucleic acid sample detection device including 1^{st} to n^{th} positive control immobilization region.

### Second Embodiment

Further, the present invention provides, as a second embodiment, a method of detecting a plurality of nucleic acid samples, wherein 1^{st} to n^{th} (n: a natural number of 2 or more) nucleic acid samples are detected on a nucleic acid sample detection device including 1^{st} to n^{th} positive control immobilization regions and 1^{st} to n^{th} negative control immobilization regions.

### Third Embodiment

Further, the present invention provides, as a third embodiment, a method of detecting a plurality of nucleic acid samples, wherein 1^{st} to n^{th} (n: a natural number of 2 or more) nucleic acid samples are detected on a nucleic acid sample detection device including 1^{st} to n^{th} positive control immobilization regions and 1^{st} to n^{th} negative control immobilization regions, wherein each negative control immobilization region is composed of 1 immobilization region.

### Fourth Embodiment

The present invention provides, as a fourth embodiment, a method of detecting a plurality of nucleic acid samples, wherein 1^{st} to n^{th} (n: a natural number of 2 or more) nucleic acid samples are detected on a nucleic acid sample detection device including 1^{st} to n^{th} positive control immobilization regions and 1^{st} to n^{th} negative control immobilization regions, by using 1^{st} to n^{th} nucleic acid sample discrimination reagents containing 1^{st} to n^{th} positive control judgment reagents and 1^{st} to n^{th} negative control judgment reagents.

### Fifth Embodiment

The present invention provides, as a fifth embodiment, a method of detecting a plurality of nucleic acids, wherein 1^{st} to n^{th} (n: a natural number of 2 or more) nucleic acid samples are detected on a nucleic acid sample detection device including 1^{st} to n^{th} positive control immobilization regions and 1^{st} to n^{th} negative control immobilization regions, by using 1^{st} to n^{th} nucleic acid sample discrimination reagents containing 1^{st} to n^{th} positive control judgment reagents and 1^{st} to n^{th} negative control judgment reagents, wherein the negative control judgment reagents have the function as a linker.

According to the present invention, there can be realized a detection method that can prevent false detection by mix-up of samples and contamination among samples and is endowed with high safety and reliability required in the clinical field.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a nucleic acid sample detection device 11 in a first embodiment.

FIG. 2 is a schematic diagram showing a method of detecting a plurality of nucleic acids by using the nucleic acid sample detection device 11 shown in FIG. 1.

FIG. 3 is a schematic diagram showing a nucleic acid sample detection device 31 in a second embodiment.

FIG. 4 is a schematic diagram showing a method of detecting a plurality of nucleic acids by using the nucleic acid sample detection device 31 shown in FIG. 3.

FIG. 5 is a schematic diagram showing a nucleic acid sample detection device 51 in a third embodiment.

FIG. 6 is a schematic diagram showing nucleic acid probes immobilized on the negative control immobilization region shown in FIG. 5.

FIG. 7 is a schematic diagram showing a method of detecting a plurality of nucleic acids by using the nucleic acid sample detection device 51 shown in FIG. 5.

FIG. 8 is a schematic diagram showing a nucleic acid sample detection device 81 in a fourth embodiment.

FIG. 9 is a schematic diagram showing a method of detecting a plurality of nucleic acids by using the nucleic acid sample detection device 81 shown in FIG. 8.

FIG. 10 is a schematic diagram showing a method of detecting a plurality of nucleic acids in a fifth embodiment.

FIG. 11 is a schematic diagram showing plural types of nuclei acids contained in negative control judgment reagents shown in FIG. 10.

FIG. 12 is a first bar graph showing current values detected from samples S1 to S4 in the first embodiment.

FIG. 13 is a second bar graph showing current values detected from samples S1 to S4 in the first embodiment.

FIG. 14 is a bar graph showing current values detected from samples S1 to S4 in the second embodiment.

FIG. 15 is a bar graph showing current values detected from samples S1 to S4 in the third embodiment.

FIG. 16 is a first bar graph showing current values detected from samples S1 to S4 in the fourth embodiment.

FIG. 17 is a second bar graph showing current values detected from samples S1 to S4 in the fourth embodiment.

FIG. 18 is a bar graph showing current values detected from samples S1 to S4 in the fifth embodiment.

### DETAILED DESCRIPTION OF THE INVENTION

### Basic Constitution

Hereinafter, (1) a nucleic acid sample detection device, (2) detection technique, (3) nucleic acid sample, and (4) detection procedures are described.

### (1) Nucleic Acid Sample Detection Device

The nucleic acid sample detection device in this embodiment is characterized by comprising, for example, a substrate, a plurality of nucleic acid probe immobilization regions formed on the substrate, and a frame for dividing the nucleic acid probe immobilization regions. The frame forms at least one well, each well constitutes one examination lane for detecting 1 nucleic acid sample, and a nucleic acid probe immobilization region on which a nucleic acid probe as a subject of examination is immobilized (hereinafter referred to as detection nucleic acid probe immobilization region) is formed in each well.

Materials used for the substrate and frame are not particularly limited and may be any materials known by those skilled in the art. The materials that can be used herein include, for example, inorganic insulating materials such as glass, quartz glass, silicon, alumina, sapphire, forsterite, silicon carbide, silicon oxide, and silicon nitride. Other examples of the materials that can be used herein include organic materials such as polyethylene, ethylene, polypropylene, polyisobutylene, polyethylene terephthalate, unsaturated polyester, fluoroplastic, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile copolymer, acrylonitrile-butadiene-styrene copolymer, silicone resin, polyphenylene oxide, and polysulfone. The shape of the substrate is not particularly limited and may be flat or uneven or may be porous.

The nucleic acid probe immobilization region is composed of a detection nucleic acid probe immobilization region, a positive control immobilization region and a negative control immobilization region.

The “detection nucleic acid probe immobilization region” is a region for detecting the presence or absence of a target nucleic acid sequence to be examined. For example, when the presence or absence of a certain disease gene is examined, the presence of absence of the disease gene in a nucleic acid sample can be judged by previously immobilizing a nucleic acid probe having a sequence complementary to the disease gene. By arranging a plurality of detection nucleic acid probe immobilization regions and immobilizing, on each region, a nucleic acid probe containing a different nucleotide sequence, a plurality of detection items can be simultaneously examined. The “nucleic acid probe having a sequence complementary to the disease gene” refers to a nucleic acid probe having a sequence complementary to at least a part of the disease gene.

The “positive control immobilization region” is a region for confirming the presence or absence of mix-up of nucleic acid samples. This “positive control immobilization region” is not present in conventional nucleic acid sample detection devices, and therefore, even if a certain nucleic acid sample is mistaken for another nucleic acid sample in detecting a plurality of nucleic acid samples simultaneously, this fact cannot be confirmed in the conventional detection devices. On the other hand, the nucleic acid sample detection device in this embodiment can confirm the presence or absence of mix-up of nucleic acid samples by the “positive control immobilization region” and can thus serve as a nucleic acid sample detection device endowed with high safety and reliability without mix-up of nucleic acid samples.

The “negative control immobilization region” is a region for confirming the presence or absence of contamination. This “negative control immobilization region” is not present in conventional nucleic acid sample detection devices, and therefore, even if a certain nucleic acid sample is contaminated with another nucleic acid sample in detecting a plurality of nucleic acid samples simultaneously, this fact cannot be confirmed in the conventional detection devices. On the other hand, the nucleic acid sample detection device in this embodiment can confirm the presence or absence of contamination with another nucleic acid sample by the “negative control immobilization region” and can thus serve as a nucleic acid sample detection device endowed with high safety and reliability without contamination.

The material for the nucleic acid probe immobilized on the nucleic acid probe immobilization region is not particularly limited and may be any material known by those skilled in the art. For example, DNA, RNA, PNA, a nucleic acid of methylphosphonate skeleton, other nucleic acid analogues, and chimeric nucleic acids thereof can be used. The length of the probe used is not particularly limited. For example, the probe is 8 to 200 bases in length, preferably 10 to 100 bases, more preferably 12 to 50 bases.

The method of immobilizing nucleic acid probes to prepare the nucleic acid probe immobilization region is not particularly limited, and any method known by those skilled in the art can be used in immobilization. The nucleic acid probe can be immobilized, for example, by physical adsorption, chemical adsorption, hydrophobic bonding, embedding, or covalent bonding. Specifically, a condensing agent such as carbodiimide can be used in covalently bonding a probe to a substrate via an amino group introduced into the end of the probe. Alternatively, by coating a substrate with an anionic organic substance, a nucleic acid probe can be immobilized on the substrate via ionic bonding. When biotin is introduced into the end of a probe, the probe can also be immobilized via biotin-avidin bonding. Further, a probe can be strongly immobilized by introducing a thiol group into the end of the probe and then forming S—S between the thiol group and a thiol-containing substance coated on a substrate. In these cases, the surface of a substrate can be previously modified with a molecule having a functional group to facilitate immobilization. For reducing the steric hindrance between the surface of the substrate and the probe, a spacer is desirably interposed between the probe and the terminal functional group. The spacer molecule is not particularly limited and may be for example an alkane skeleton, an alkyne skeleton, an alkene skeleton, an ethylene glycol skeleton or a nucleic acid chain. Its molecular structure may be either a linear chain or a branched chain. The length of the spacer is not particularly limited, but is preferably 10 to 500, more preferably 20 to 200, even more preferably 50 to 100, in terms of the number of carbon-carbon bonds.

When an immobilization device called a DNA spotter or a DNA arrayer is used in immobilization of the nucleic acid probe on a substrate, the probe can relatively easily be immobilized. In this case, a spotter of an ink jet system or an electrostatic system is preferably used to prevent the surface of the substrate from being damaged. It is also possible to directly synthesize the nucleic acid chain on the surface of the substrate.

The process of immobilizing nucleic acid probes to form the nucleic acid probe immobilization region is carried out preferably before bonding a substrate to a frame, but even after bonding, nucleic acid probes can be immobilized. The nucleic acid sample detection device in this embodiment is not necessarily provided in the state where nucleic acid probes have been immobilized on the nucleic acid probe immobilization region, and the device may be provided as a substrate on which nucleic acid probes are not immobilized. In this case, desired nucleic acid probes are immobilized on the substrate as described above so that the substrate can be used as the nucleic acid sample detection device.

The shape of a well formed by a frame is also not particularly limited and may be circular, rectangular or polygonal. The nucleic acid detection substrate in this embodiment would be easily prepared by preparing a frame with wells having no bottom and then bonding the frame to a substrate on which the nucleic acid probe immobilization region has been formed for use. For bonding the frame to the substrate, a strong bonding method such as adhesive bonding or pressure bonding to prevent liquid leakage is desired, and a silicon packing can also be used in bonding.

The nucleic acid detection substrate in this embodiment does not always have to be provided in the state where the substrate on which the nucleic acid probe immobilization region has been formed and the frame are integrated into one body, and the nucleic acid detection substrate may be provided in the state where both the substrate and the frame are separate. In this case, the substrate and the frame are bonded as described above for use as the nucleic acid detection substrate.

The nucleic acid sample detection device in this embodiment is provided with a plurality of wells formed by the frame in order to detect a plurality of nucleic acid samples. The number of wells is not particularly limited, and is preferably 3 or more and less than 100, more preferably 4 or more and 50 or less, still more preferably 5 or more and 20 or less. These wells are formed on the same device but are not always formed on 1 substrate. All wells may be formed on 1 substrate, or a plurality of wells may be formed on a plurality of substrates. For example, a plurality of nucleic acid samples may be detected simultaneously by preparing a plurality of substrates each having 1 well formed thereon. In this case, 1 nucleic acid sample is detected with 1 substrate.

### (2) Detection Technique

The present invention includes all detection techniques known to those skilled in the art, and should not be construed as restrictive with respect to detection techniques. For example, detection by the fluorescence intensity of a fluorescent dye, detection by a radioisotope, detection by an electrochemical response of an intercalator molecule, and detection by a change in electrostatic capacity can be used. Particularly, as typical detection techniques, there are detection by fluorescence and electrochemical detection. In detection by fluorescence, detection results can be visually recognized, thus preventing erroneous judgment of the results. In electrochemical detection, on the other hand, only current values are used in examination, and thus the device can be downsized, the examination cost can be reduced, and the examination time can be shortened. In the case of electrochemical detection, each of the nucleic acid probe immobilization regions is constituted as an electrode, and each nucleic acid probe is immobilized on the electrode.

In the case of fluorescence detection, the nucleic acid sample is previously labeled with a fluorescent substance. For example, a primer labeled with a fluorescent substance is used to amplify the target nucleic acid by PCR. The target nucleic acid that formed a hybrid chain with a nucleic probe remains in the nucleic acid probe immobilization region even after washing and thus gives fluorescence light. The fluorescent substance used may be an arbitrary fluorescent substance known in the art, and for example, FITC, Cy3, Cy5 or rhodamine is used. The emission of the fluorescent substance can be detected with a fluorescence detector. The presence or absence of the target nucleic acid corresponding to each nucleic acid probe can be determined from the amount of the obtained fluorescence.

For preventing the unspecific adsorption of nucleic acids or fluorescent dyes onto the nucleic acid probe immobilization region in the fluorescence detection method, the surface of the nucleic acid probe immobilization region is desirably coated with lipids, surfactants, albumins or nucleic acids, such as mercaptoethanol, mercaptohexanol, mercaptoheptanol, mercaptoethylene glycol, mercaptooligoethylene glycol, mercaptopolyethylene glycol, mercaptans such an alkane thiol having a C30 to C50 chain, and stearylamines.

In the case of electrochemical detection, on the other hand, an electrochemically active molecule is used. The electrochemically active molecule refers to a molecule which binds to a hybrid chain and emits an electron upon application of an electric potential. An arbitrary electrochemically active molecule known in the art may be used. Examples of the electrochemically active molecule that can be used include Hoechst 33258 (registered trademark) (available from CALBIOCHEM), Acridine Orange, quinacrine, daumonycin, a metallo-intercalator, a bis-intercalator such as bisacridine, a tris-intercalator or a poly-intercalator. Particularly, Hoechst 33258 (registered trademark) is preferably used. Hoechst 33258 (registered trademark) is a molecule composed of a chemical substance p-(5-(5-(4-methylpiperazin-1-yl)benzimidazol-2-yl) benzimidazol-2-yl) phenol. Moreover, these intercalators may be modified with an electrochemically active metal complex such as ferrocene (dicyclopentadienyl iron) or viologen. The concentration of the molecule is selected appropriately, and is generally in the range of from 1 ng/ml to 1000 ng/ml. At this time, a buffer solution having an ionic strength ranging from 0.01 to 5 and a pH ranging from 5 to 10 can be used.

The molecule recognizes the hybrid chain and intercalates it. Upon application of a potential, the redox reaction of the molecule occurs to release an electron therefrom, thus bringing about passage of a current. Thereupon, the potential may be swept at a constant rate or applied by pulsation, or a constant potential may be applied. The potential sweeping rate is in the range of 10 to 1000 mV/sec. For measurement, the electricity and voltage may be regulated by using a device such as a potentiostat, a digital multimeter and a function generator. A current derived from the molecule flows in the electrode, and the presence or absence of the target nucleic acid corresponding to each nucleic acid probe can be determined by measuring the current value.

For preventing the unspecific adsorption of nucleic acids or intercalators onto the nucleic acid probe immobilization region (electrode) in the electrochemical detection method, the surface of the electrode is desirably coated with lipids, surfactants, albumins or nucleic acids, such as mercaptoethanol, mercaptohexanol, mercaptoheptanol, mercaptoethylene glycol, mercaptooligoethylene glycol, mercaptopolyethylene glycol, mercaptans such as an alkane thiol having a C30 to C50 chain, and stearylamines.

### (3) Nucleic Acid Sample

The nucleic acid sample detected by the nucleic acid sample detection device in this embodiment is not especially limited, and may be a nucleic acid sample extracted from a sample such as blood, serum, leukocyte, urine, feces, semen, salivary juice, tissue, cultivated cell, phlegm, food, soil, drainage, waste water, air, and the like, or a nucleic acid sample obtained by amplification treatment after extraction. The detection method in this embodiment can be used to detect, for example, virus infections caused by viruses such as hepatitis virus (A, B, C, D, E, F, and G types), HIV, influenza virus (A, B, C, D, E, and F), herpes group virus, adenovirus, human polyoma virus, human papilloma virus, human parvovirus, mumps virus, human rotavirus, enterovirus, Japanese encephalitis virus, smallpox virus, coronavirus, SARS, dengue fever virus, rubella viruses, and HTLV, infections caused by microorganisms such as yellow staphylococcus, hemolytic streptococcus, pathogenic Escherichia coli, enteritis vibrio, Helicobacter pylori, campylobacter, cholera bacterium, dysentery bacterium, salmonella, anthrax, yersinia, gonococcus, listeria, leptospire, legionalla bacterium, spirochete, pneumonia mycoplasma, rickettsia, chlamydia, malaria, dysentery amoebas, and pathogenetic fungus as well as parasite and fungus.

The detection method in this embodiment can also be used in genotyping of microorganisms causing the infections mentioned above. For example, the detection method can be used in detecting genotypes of the HCV virus, that is, 1a, 1b, 2a, 2b, 3a and 3b and genotypes of human papillomavirus, that is, 16, 18, 31, 33, 35, 39, 45, 51, 52, 53, 54, 56, 58, 59, 66, 68 and 69 which are related to malignant transformation and 6, 11, 34, 40, 42, 43, 44 and 70 which are not related to malignant transformation. Drug resistance genes can also be detected, and examples include drug resistance genes of the tubercle bacillus, AIDS virus, and microorganisms causing respiratory infections. The detection method in this embodiment can also be used in examining hereditary diseases, neoplastic diseases such as retinoblastoma, virus tumor, familial colonic polyposis, hereditary nonpolyposis colon cancer, neurofibromatosis, familial chest cancer, xeroderma pigmentosum, brain cancer, oral cancer, esophageal cancer, stomach cancer, colon cancer, liver cancer, pancreatic cancer, lung cancer, thyroid tumor, mammary gland tumor, urinary tumor, virilia tumor, muliebria tumor, skin tumor, osteosarcoma, osteochondrosarcoma, leukemia, lymphoma, and solid tumor. The detection method can be adopted to all fields to which the gene check is necessary; in a food check, quarantine, medicine check, legal medicine, agriculture, stock raising, fishery, and forestry, etc. as well as in medical treatment. In addition, the detection of restriction fragment length polymorphism (RFLP), single nucleotide polymorphisms (SNPs), and the micro satellite array, etc. is also possible. The detection method can also be used for analyzing unknown nucleotide sequences.

### (4) Detection Procedures

For detection of nucleic acids contained in samples, a nucleic acid component is extracted from the samples thereby obtaining nucleic acid samples. There is no particular limitation to the extraction method, and a liquid-liquid extraction such as a phenol/chloroform method or a solid-liquid extraction using a carrier may also be used. A commercially available nucleic acid extraction method such as a QIAamp method (produced by QIAGEN) or Sumi Test (produced by Sumitomo Metal Industries, Ltd.) can also be utilized. These samples are pipetted onto a microtiter plate or the like and subjected to gene detection. When a microtiter plate retaining a hydrophobic membrane, for example, is used in gene extraction, a detection operation can be started more easily. The extracted nucleic acid is preferably dissolved in a suitable solution.

The hybridization reaction between the nucleic acid and a probe immobilized on a probe immobilization region is carried out in a buffer solution as a reaction solution having an ionic strength ranging from 0.01 to 5 and a pH ranging from 5 to 10. Dextran sulfate, salmon sperm DNA, bovine thymus DNA, EDTA and surfactants may be added as hybridization accelerators to the reaction solution. Further, a salt concentration regulator, a positive control reagent, etc. may also be added. According to necessity, a nucleic acid amplification reaction may be carried out as a pretreatment. The nucleic acid amplification reaction includes, but is not limited to, PCR, LAMP, and ICAN. Then, the extracted nucleic acid is added to the reaction solution and thermally denatured at 90° C. or more. Contacting the nucleic acid-containing reaction solution with a probe immobilization electrode may be carried out immediately after the denaturation or after rapid cooling to 0° C. During the reaction, the reaction rate can be increased by an operation such as stirring or shaking. The reaction may be performed at a temperature ranging from 10 to 90° C. for the period of about one minute to overnight. After the hybridization reaction, the probe immobilization region is washed. In washing, a buffer solution having an ion strength ranging from 0.01 to 5 and a pH ranging from 5 to 10 is used. At this time, a nucleic acid sample discrimination reagent is added to the nucleic acid-containing reaction solution. The composition of the nucleic acid sample discrimination reagent depends on whether the immobilized probe is a positive control nucleic acid probe or a negative control nucleic acid probe.

After washing, the presence or absence of hybridization is detected. The detection techniques are not particularly limited, and detection by the fluorescence intensity of a fluorescent dye, detection by a radioisotope, detection by an electrochemical response of an intercalator molecule, detection by a change in electrostatic capacity, or the like, can be used as described above.

According to the detection method in this embodiment, samples derived from different analytes are used in the respective reaction wells formed on the substrate, and nucleic acid chains in the samples can be simultaneously detected.

The nucleic acid reaction and nucleic acid detection can be automated. Variations in measurement result, which are attributable to handling, etc., can be reduced by automation. The automated examination apparatus can include a temperature regulation system for regulating the reaction temperature in an extraction reaction, an amplification reaction, a hybridization reaction and a washing reaction. A Peltier element, an electrothermal heater, etc. may be utilized in the temperature regulating system. The automated examination apparatus can also include a solution-sending system for sending a solution of each reagent. In the solution-sending system, a pump, a pipe, a flow-rate monitor, a degassing mechanism, a gas/liquid detection monitor, or the like, can be used. The automated examination apparatus can also include a detection system for detecting a double-stranded nucleic acid and a single-stranded nucleic acid. Although the detection system varies depending on the detection method, a laser irradiation device and CCD camera can be used in the fluorescence detection method. A two- or three-electrode current/voltage regulating device can be used in the current detection method. The automated examination apparatus can also include a signal processing system for performing automatic judgment based on an obtained signal. Using a database, a threshold value, etc. previously incorporated in a computer, a sample nucleic acid can be sequenced, or the presence or absence of a target nucleic acid can be judged on the basis of an obtained signal. The automated apparatus can deal with a plurality of nucleic acid sample detection devices at one time. One apparatus may also perform the entire process, or a plurality of apparatuses may share the process.

### DETAILED DESCRIPTION OF THE PREFERRED EMBODIMENTS

Hereinafter, embodiments of the present invention are described with reference to the drawings. The embodiments shown below are set forth for illustrative purposes for describing the constitution of the invention in detail. Therefore, the invention should not be construed as restrictive on the basis of the following description of embodiments. The scope of the invention encompasses all embodiments which are within the scope of the invention as defined by the appended claims, and include a wide variety of alterations and modifications thereof.

### (1) First Embodiment

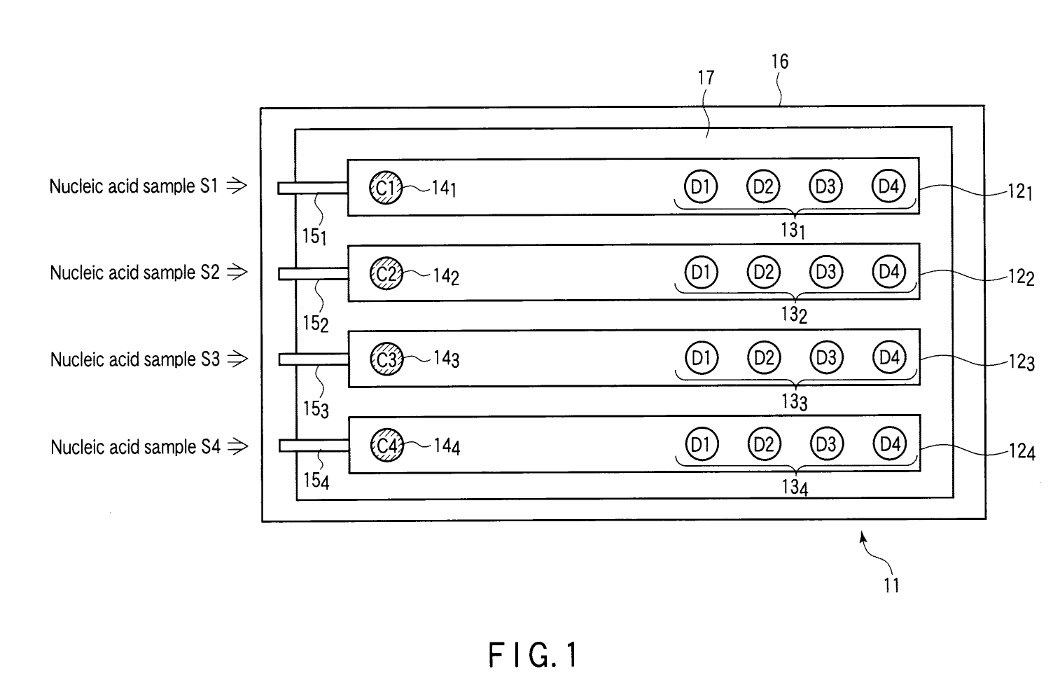

### FIG. 1

FIG. 1 is a schematic diagram showing a nucleic acid sample detection device 11 in a first embodiment. The nucleic acid sample detection device 11 is characterized by comprising a substrate 16, a plurality of nucleic acid probe immobilization regions formed on the substrate, and a frame 17 for dividing the nucleic acid probe immobilization regions. The frame 17 forms one or more wells 12, each well constitutes one examination lane for detecting one nucleic acid sample, and detection nucleic acid probe immobilization regions 13 on which a nucleic acid probe as the subject of examination has been formed are formed in the wells 12, respectively.

1^{st} to n^{th} wells 12₁₋ₙ are provided with injection ports 15₁₋ₙ (also referred to hereinafter as 1^{st} to n^{th} injection ports 15₁₋ₙ) for injection of 1^{st} to n^{th} nucleic acid samples respectively. The 1^{st} well 12₁ is a well for detecting the first nucleic acid sample, and the k^{th} (k: a natural number of 2 or more) well 12ₖ is a well for detecting the k^{th} nucleic acid sample.

The 1^{st} well 12₁ includes a detection nucleic acid probe immobilization region 13₁ for detecting the 1^{st} nucleic acid sample (also referred to hereinafter as the 1^{st} detection nucleic acid probe immobilization region 13₁) and a positive control immobilization region 14₁ for discriminating the 1^{st} nucleic acid sample (also referred to hereinafter as the 1^{st} positive control immobilization region 14₁), while the k (k: a natural number of 2 to n) well 12ₖ includes a nucleic acid probe immobilization region 13ₖ for detecting the k^{th} nucleic acid sample (also referred to hereinafter as the k^{th} detection nucleic acid probe immobilization region 13ₖ) and a positive control immobilization region 14ₖ for discriminating the k^{th} nucleic acid sample (also referred to hereinafter as the k^{th} positive control immobilization region 14ₖ). The nucleic acid sample detection device 11 constituted as described can detect the 1^{st} to n^{th} (n: a natural number of 2 or more) nucleic acid samples.

A nucleic acid sample detection device 11 capable of detecting 1^{st} to 4^{th} (n=4) nucleic acid samples (nucleic acid samples S1 to S4), for example, is shown in FIG. 1. 1^{st} to 4^{th} wells 12₁₋₄ are provided with 1^{st} to 4^{th} injection ports 15₁₋₄ for injecting the 1^{st} to 4^{th} nucleic acid samples, respectively.

Discrimination nucleic acid probes different from one another are immobilized on the “positive control immobilization regions” respectively. For example, a nucleic acid probe C1 constituting the 1^{st} positive control is immobilized on the 1^{st} positive control immobilization region 14₁, a nucleic acid probe C2 constituting the 2^{nd} positive control is immobilized on the 2^{nd} positive control immobilization region 14₂, a nucleic acid probe C3 constituting the 3^{rd} positive control is immobilized on the 3^{rd} positive control immobilization region 14₃, and a nucleic acid probe C4 constituting the 4^{th} positive control is immobilized on the 4^{th} positive control immobilization region 14₄.

In the “detection nucleic acid probe immobilization region”, one or more nucleic acid probes complementary to target sequences to be detected are immobilized on mutually independent regions, respectively. For example, when there are 2 or more target sequences to be detected, the detection nucleic acid probe immobilization region is provided with 2 or more independent immobilization regions, and on each of the immobilization regions, a nucleic acid probe complementary to one of the target sequences is immobilized. FIG. 1 shows the case where the number of target sequences to be detected is 4, and the detection nucleic acid probe immobilization region in each well is provided with 4 independent immobilization regions, and nucleic acid probes D1, D2, D3 and D4, each of which is complementary to one of the target sequences, are immobilized on each of the immobilization regions. The “target sequence” used herein means, for example, one having a sequence complementary to at least a part of e.g. a disease gene to be examined. Generally, attention is focused on a sequence site characteristic of a gene to be examined, a nucleic acid probe containing a sequence complementary to this sequence site is designed, and this probe is immobilized on a detection nucleic acid probe immobilization region, whereby the target gene is detected.

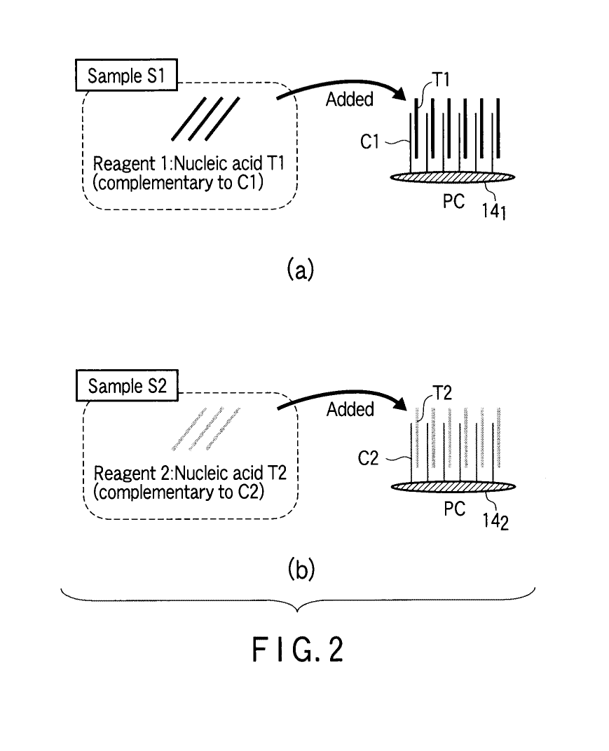

### FIG. 2

FIG. 2 is a schematic diagram showing a method of detecting a plurality of nucleic acids by using the nucleic acid sample detection device 11 shown in FIG. 1.

1^{st} to n^{th} (n: a natural number of 2 or more) nucleic acid sample discrimination reagents are added to the 1^{st} to n^{th} nucleic acid samples, respectively. The 1^{st} nucleic acid sample discrimination reagent contains a “1^{st} positive control nucleic acid” having a sequence complementary to a nucleic acid probe C1 immobilized on the 1^{st} positive control immobilization region 14₁. The k^{th} (k: a natural number of 2 or more) nucleic acid sample discrimination reagent contains a k^{th} positive control nucleic acid having a sequence complementary to a nucleic acid probe C(k) immobilized on the k^{th} positive control immobilization region 14ₖ. The 1^{st} to n^{th} nucleic acid samples to which the 1^{st} to n^{th} nucleic acid sample discrimination reagents including the above components were added respectively are injected respectively via the 1^{st} to n^{th} injection ports 15₁₋ₙ disposed in the 1^{st} to n^{th} wells, thereby causing hybridization reactions in the 1^{st} to n^{th} positive control immobilization regions 14ₙ₋₁, and by detecting the reactions, it can be confirmed that there was no mix-up of the 1^{st} to n^{th} nucleic acid samples.

In FIG. 2, the 1^{st} nucleic acid sample discrimination reagent (reagent 1) is added to the 1^{st} nucleic acid sample (sample S1) (FIG. 2a), and the 2^{nd} nucleic acid sample discrimination reagent (reagent 2) is added to the 2^{nd} nucleic acid sample (sample S2) (FIG. 2b). Similarly, the 3^{rd} nucleic acid sample discrimination reagent (reagent 3) is added to the 3^{rd} nucleic acid sample (sample S3), and the 4^{th} nucleic acid sample discrimination reagent (reagent 4) is added to the 4^{th} nucleic acid sample (sample S4).

The 1^{st} nucleic acid sample discrimination reagent (reagent 1) contains a nucleic acid T1 having a sequence complementary to the nucleic acid probe C1 immobilized on the 1^{st} positive control (PC) immobilization reagent 14₁ (FIG. 2(a)), and the 2^{nd} nucleic acid sample discrimination reagent (reagent 2) contains a nucleic acid T2 having a sequence complementary to the nucleic acid probe C2 immobilized on the 2^{nd} positive control (PC) immobilization reagent 14₂ (FIG. 2(b)). Similarly, the 3^{rd} nucleic acid sample discrimination reagent (reagent 3) contains a nucleic acid T3 having a sequence complementary to the nucleic acid probe C3 immobilized on the 3^{rd} positive control immobilization reagent 14₃, and the 4^{th} nucleic acid sample discrimination reagent (reagent 4) contains a nucleic acid T4 having a sequence complementary to the nucleic acid probe C4 immobilized on the 4^{th} positive control immobilization reagent 14₄.

The 1^{st} to 4^{th} nucleic acid samples (samples S1 to S4) to which the 1^{st} to 4^{th} nucleic acid sample discrimination reagents (reagents 1 to 4) were added are injected into the 1^{st} to 4^{th} wells 14₁ to 14₄, respectively. The samples are injected via the 1^{st} to 4^{th} injection ports 15₁ to 15₄ disposed respectively in the 1^{st} to 4^{th} wells 14₁ to 14₄. For example, when the 1^{st} nucleic acid sample to which the 1^{st} nucleic acid sample discrimination reagent (reagent 1) was added is injected into the 1^{st} well 12₁, a nucleic acid T1 contained in the reagent 1 hybridizes with the nucleic acid probe C1 immobilized on the 1^{st} positive control immobilization region 14₁ (FIG. 2(a)). As a result, a signal derived from the formed hybrid chain can be detected. When the 1^{st} nucleic acid is erroneously injected into the 2^{nd} well 12₂, a nucleic acid T1 contained in the reagent 1 cannot hybridize with the nucleic acid probe C2 immobilized on the 2^{nd} positive control immobilization region 14₂. As a result, no signal derived from the formed hybrid chain can be detected, and it can thus be reliably and easily confirmed that there was a mix-up of the samples.

### (2) Second Embodiment

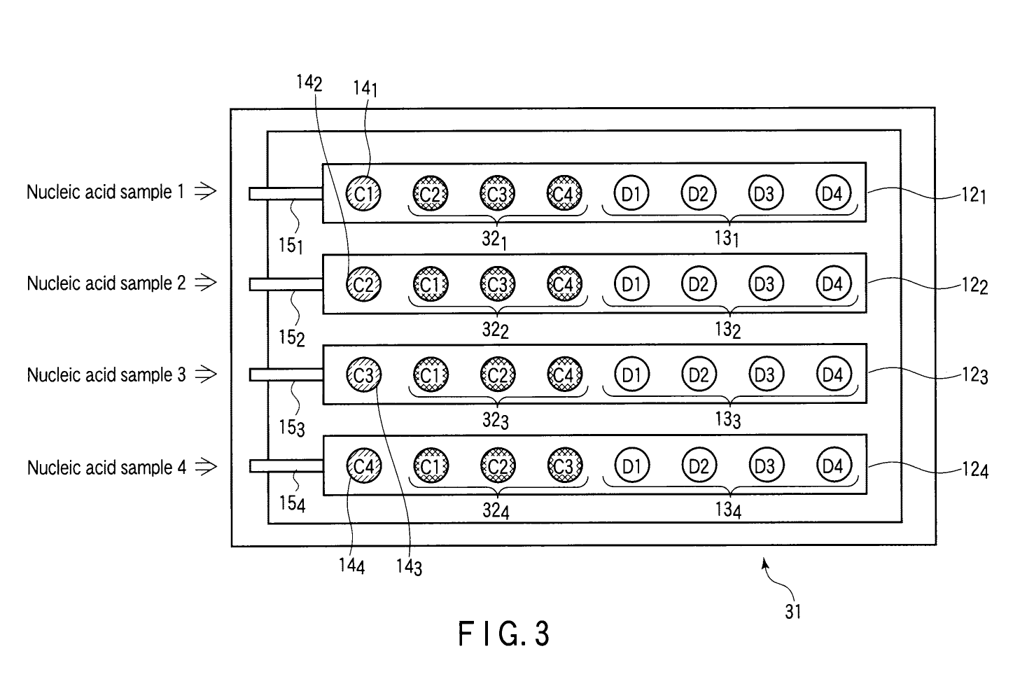

### FIG. 3

FIG. 3 is a schematic diagram showing a nucleic acid sample detection device 31 in a second embodiment. The nucleic acid sample detection device 31 in the second embodiment includes 1^{st} to n^{th} negative control immobilization regions 32₁₋ₙ in addition to positive control immobilization regions 14ₙ₋₁ in the wells.

The 1^{st} negative control immobilization region 32₁ is composed of (n-1) immobilization regions on which the nucleic acid probes C2 to Cn immobilized on the 2^{nd} to n^{th} positive control immobilization regions have been immobilized respectively and independently, and the k^{th} (k: a natural number of 2 to n) negative control immobilization region 32ₖ is composed of (n-1) immobilization regions on which the nucleic acid probes C1 to Cn immobilized on the 1^{st} to n^{th} positive control immobilization regions, excluding the nucleic acid probe C(k) immobilized on the k^{th} positive control immobilization region, have been immobilized respectively and independently.

A nucleic acid sample detection device 31 capable of detecting 1^{st} to 4^{th} (n=4) nucleic acid samples, for example, is shown in FIG. 3. The 1^{st} negative control immobilization region 32₁ is composed of 3 immobilization regions on which the nucleic acid probes C2, C3 and C4 immobilized on the 2^{nd}, 3^{rd} and 4^{th} positive control immobilization regions have been immobilized respectively and independently, the 2^{nd} negative control immobilization region 32₂ is composed of 3 immobilization regions on which the nucleic acid probes C1, C3 and C4 immobilized on the 1^{st}, 3^{rd} and 4^{th} positive control immobilization regions have been immobilized respectively and independently, the 3^{rd} negative control immobilization region 32₃ is composed of 3 immobilization regions on which the nucleic acid probes C1, C2 and C4 immobilized on the 1^{st}, 2^{nd} and 4^{th} positive control immobilization regions have been immobilized respectively and independently, and the 4^{th} negative control immobilization region 32₄ is composed of 3 immobilization regions on which the nucleic acid probes C1, C2 and C3 immobilized on the 1^{st}, 2^{nd} and 3^{rd} positive control immobilization regions have been immobilized respectively and independently.

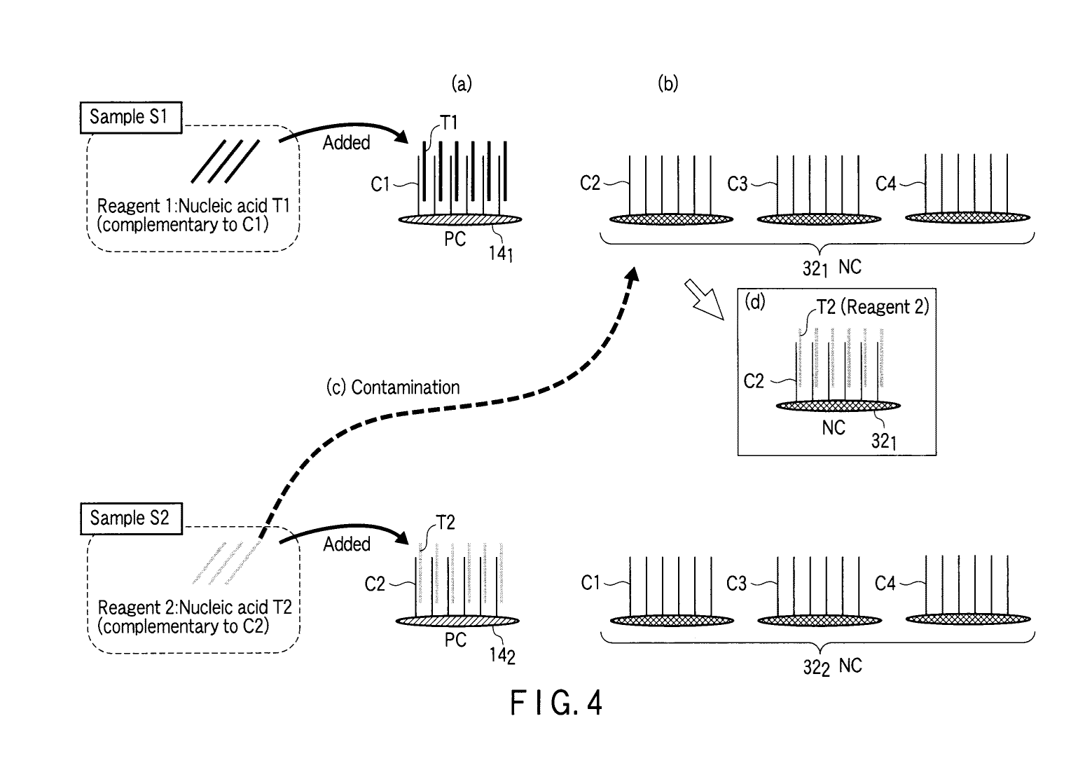

### FIG. 4

FIG. 4 is a schematic diagram showing a method of detecting a plurality of nucleic acids by using the nucleic acid sample detection device 31 shown in FIG. 3. The difference between the second embodiment and the first embodiment is that not only mix-up of nucleic acid samples but also contamination of a nucleic acid sample with another nucleic acid sample can be detected by arranging negative controls in each well.

Similarly to the first embodiment, the 1^{st} to 4^{th} nucleic acid samples (samples S1 to S4) to which the 1^{st} to 4^{th} nucleic acid sample discrimination reagents (reagents 1 to 4) were added are injected into the 1^{st} to 4^{th} wells 12₁ to 12₄ respectively. The samples are injected respectively via the 1^{st} to 4^{th} injection ports 15₁ to 15₄ disposed in the 1^{st} to 4^{th} wells 12₁ to 12₄. For example, when the 1^{st} nucleic acid sample to which the 1^{st} nucleic acid sample discrimination reagent (reagent 1) was added is injected into the 1^{st} well 12₁, a nucleic acid T1 contained in the reagent 1 hybridizes with the nucleic acid probe C1 immobilized on the 1^{st} positive control immobilization region 14₁ (FIG. 4(a)). As a result, a signal derived from the formed hybrid chain can be detected, while the nucleic acid T1 contained in the reagent 1 does not hybridize with any nucleic acid probes C2, C3 and C4 immobilized on the 1^{st} negative control immobilization region 32₁ (FIG. 4(b)). As a result, a signal is not detected from the 1^{st} negative control immobilization region 32₁, and it can thus be confirmed that there was no contamination of the sample with another nucleic acid sample.

On the other hand, when a signal is detected from the 1^{st} negative control immobilization region 32₁, the contamination of the sample with another nucleic acid sample can be detected. For example, when the 1^{st} nucleic acid sample (sample S1) has been contaminated with the 2^{nd} nucleic acid sample (sample S2) to which the 2^{nd} nucleic acid sample discrimination reagent (reagent 2) was added (FIG. 4(c)), the 1^{st} nucleic acid sample (sample S1) has been contaminated with the nucleic acid T2 contained in the reagent 2, and thus the nucleic acid T2 hybridizes with the nucleic acid probe C2 immobilized on the 1^{st} negative control immobilization region 32₁ (FIG. 4(d)). As a result, a signal derived from the formed hybrid chain is detected, and the contamination of the sample with another nucleic acid can be detected. In the second embodiment, not only mix-up of nucleic acid samples but also contamination can be detected, and thus a safer and highly reliable detection method can be provided.

### (3) Third Embodiment

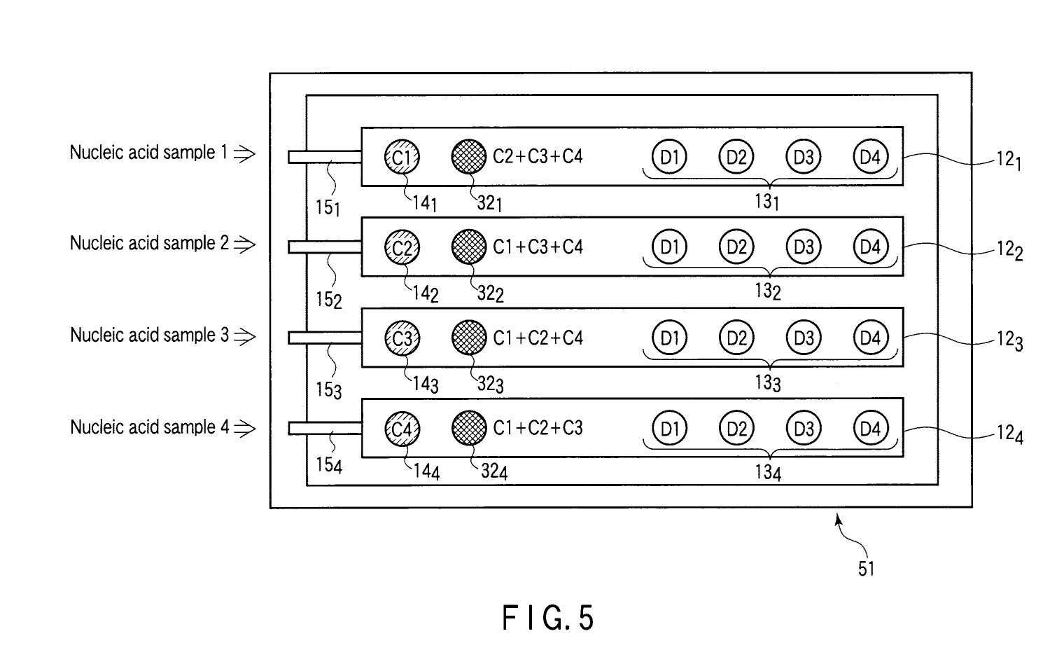

### FIG. 5

FIG. 5 is a schematic diagram showing a nucleic acid sample detection device 51 in a third embodiment.

The nucleic acid sample detection device 51 in the third embodiment is characterized in that a plurality of negative control immobilization regions arranged in each well in the second embodiment are constituted as one immobilization region.

The 1^{st} negative control immobilization region is composed of one immobilization region on which nucleic acid probes having the same sequences as in the nucleic acid probes C2 to C(n) immobilized on the 2^{nd} to n^{th} positive control immobilization regions have been immobilized together, and the k^{th} (k: a natural number of 2 to n) negative control immobilization region is composed of one immobilization region on which nucleic acid probes having the same sequences as in the nucleic acid probes immobilized on the 1^{st} to n^{th} positive control immobilization regions, excluding the nucleic acid probe C(k) immobilized on the k^{th} positive control immobilization region, have been immobilized together.

A nucleic acid sample detection device 51 capable of detecting 1^{st} to 4^{th} (n=4) nucleic acid samples for example is shown in FIG. 5. The 1^{st} negative control immobilization region 32₁ is composed of one immobilization region on which the nucleic acid probes C2, C3 and C4 immobilized on the 2^{nd}, 3^{rd} and 4^{th} positive control immobilization regions respectively have been immobilized together, the 2^{nd} negative control immobilization region 32₂ is composed of one immobilization region on which the nucleic acid probes C1, C3 and C4 immobilized on the 1^{st}, 3^{rd} and 4^{th} positive control immobilization regions respectively have been immobilized together, the 3^{rd} negative control immobilization region 32₃ is composed of one immobilization region on which the nucleic acid probes C1, C2 and C4 immobilized on the 1^{st}, 2^{nd} and 4^{th} positive control immobilization regions respectively have been immobilized together, and the 4^{th} negative control immobilization region 32₄ is composed of one immobilization region on which the nucleic acid probes C1, C2 and C3 immobilized on the 1^{st}, 2^{nd} and 3^{rd} positive control immobilization regions respectively have been immobilized together.

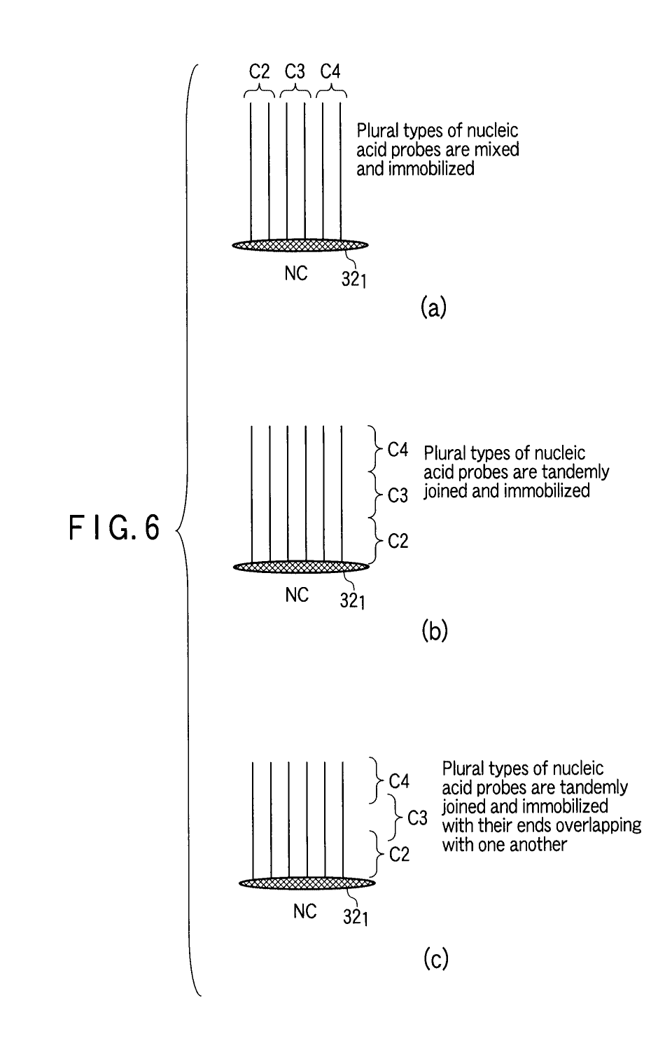

### FIG. 6

FIG. 6 is a schematic diagram showing the nucleic acid probes immobilized on the negative control immobilization region shown in FIG. 5. The difference between the third embodiment and the second embodiment is that the negative control immobilization region is composed of one region. The negative controls are different from the positive controls in that as the number of nucleic acid samples increases, the number of nucleic acid probes necessary as negative controls increases. If a plurality of nucleic acid probes are immobilized together on one immobilization region, it is not necessary for the number of negative control immobilization regions to be increased even if the number of nucleic acid samples increases, and irrespective of the number of samples, a sufficient space for examination items can be steadily secured on the substrate.

When a plurality of negative controls are arranged in one region, the arrangement includes, for example, an arrangement in which plural types of nucleic acid probes constituting negative controls are immobilized in parallel (FIG. 6(a)), an arrangement in which nucleic acid chains each consisting of plural types of tandemly joined nucleic acid probes are immobilized (FIG. 6(b)), and an arrangement in which nucleic acid chains each consisting of plural types of tandemly joined nucleic acid probes with their ends overlapping with one another are immobilized (FIG. 6(c)). In the arrangement in which plural types of nucleic acid probes are immobilized in parallel (FIG. 6(a)), the region of the negative controls may be divided into 2 or 3 regions as the number of negative controls increases. For example, when the negative controls are composed of 8 nucleic acid probes, the 8 probes are divided into 2 groups each consisting of 4 probes and the 2 groups are immobilized on 2 regions; when the negative controls are composed of 9 nucleic acid probes, the 9 probes are divided into 3 groups each consisting of 3 probes and the 3 groups are immobilized on 3 regions, and so on. When the number of negative controls further increases, the negative control regions may be divided into 4, 5 or more groups each of which may be immobilized on an independent region.

As the downsizing of an examination substrate advances at present, probe immobilization spots thereon tend to be micronized. A significant increase in the number of samples is also estimated. The increase in the number of samples leads to an increase in the number of negative controls, but when a large number of negative controls are mixed and arranged in parallel on a micronized region estimated to be realized in the future (FIG. 6(a)), the amount of each of the negative control immobilized is reduced, and consequently a sufficient signal may not be detectable. However, when a plurality of nucleic acid probes are joined tandemly and spatially developed (FIG. 6(b)), the amount of each of the negative controls immobilized can be maintained. By so doing, a large number of negative controls can be immobilized even on a minimized region estimated to be realized in the future. When negative controls are tandemly joined, the respective nucleic acid probes may be arranged so as to allow their ends to overlap with one another so that the length of the nucleic acids upon tandem joining can be prevented from increasing (FIG. 6(c)), which is particularly effective when the number of samples is increased.

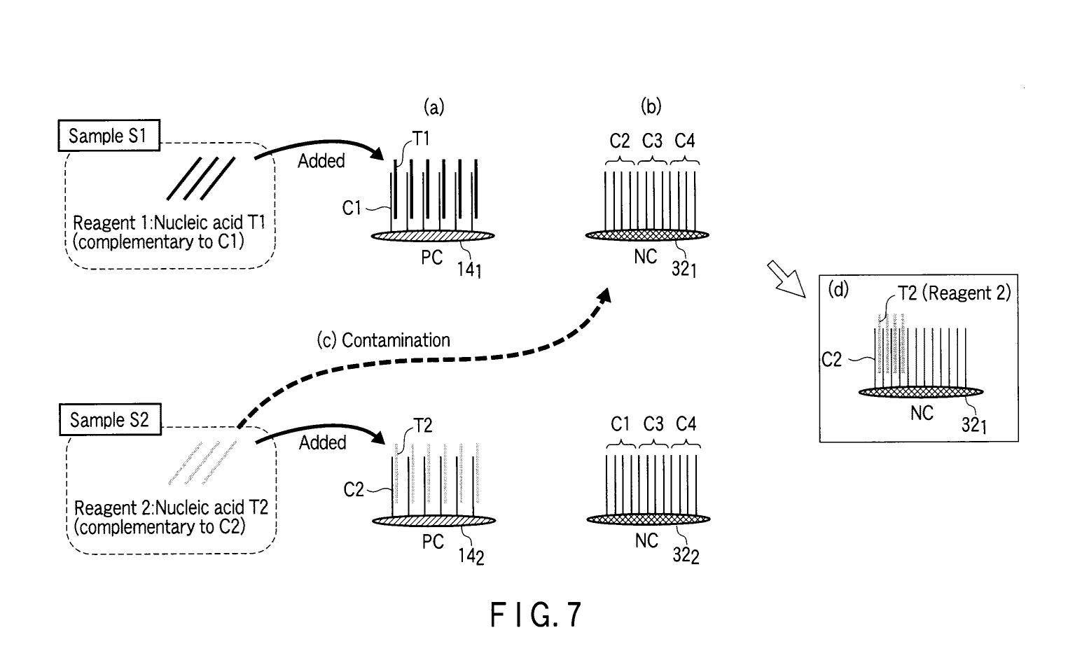

### FIG. 7

FIG. 7 is a schematic diagram showing a method of detecting a plurality of nucleic acids by using the nucleic acid sample detection device 51 shown in FIG. 5. The difference between the third embodiment and the second embodiment is that the negative control immobilization region is composed of one region. The space necessary of the negative controls on the substrate can be significantly reduced by making the number of the negative control immobilization region be 1, and this effect is made more significant as the number of samples increases.

In the third embodiment, similarly to the second embodiment, the 1^{st} to 4^{th} nucleic acid samples (samples S1 to S4) to which the 1^{st} to 4^{th} nucleic acid sample discrimination reagents (reagents 1 to 4) were added are injected into the 1^{st} to 4^{th} wells 12₁ to 12₄ respectively. The samples are injected via 1^{st} to 4^{th} injection ports 15₁ to 15₄ disposed in the 1^{st} to 4^{th} wells 12₁ to 12₄. For example, when the 1^{st} nucleic acid sample to which the 1^{st} nucleic acid sample discrimination reagent (reagent 1) was added is injected into the 1^{st} well 12₁, a nucleic acid T1 contained in the reagent 1 hybridizes with the nucleic acid probe C1 immobilized on the 1^{st} positive control immobilization region 14₁ (FIG. 7(a)). As a result, a signal derived from the formed hybrid chain can be detected, while the nucleic acid T1 contained in the reagent 1 does not hybridize with any nucleic acid probes C2, C3 and C4 immobilized on the 1^{st} negative control immobilization region 32₁ (FIG. 7(b)). As a result, no signal is detected from the 1^{st} negative control immobilization region 32₁, and it can thus be confirmed that there was no contamination with another nucleic acid sample. FIG. 7 shows the negative control immobilization region on which plural types of nucleic acid probes constituting negative controls are mixed and immobilized, but the negative immobilization region may be a region on which plural types of nucleic acid probes are tandemly joined and immobilized (FIG. 6b) or a region on which plural types of nucleic acid probes are joined tandemly with their ends overlapping with one another and immobilized (FIG. 6c).

When a signal is detected from the 1^{st} negative control immobilization region 32₁, contamination with another nucleic acid sample can be detected. For example, when the 1^{st} nucleic acid sample (sample S1) has been contaminated with the 2^{nd} nucleic acid sample (sample S2) to which the 2^{nd} nucleic acid sample discrimination reagent (reagent 2) was added (FIG. 7c), the 1^{st} nucleic acid sample has been contaminated with a nucleic acid T2 contained in the reagent 2, and thus the nucleic acid T2 hybridizes with a nucleic acid probe C2 immobilized on the 1^{st} negative control immobilization region 32₁ (FIG. 7d). As a result, a signal derived from the formed hybrid chain is detected, and contamination with another nucleic acid can be detected.

The third embodiment, similarly to the second embodiment, can detect not only mix-up of nucleic acid samples but also contamination, thus providing a safer and highly reliable detection method. In the third embodiment, the nucleic acid probes immobilized on the negative control immobilization region have been immobilized together on one region so that even if the number of nucleic acid samples increase, it is not necessary to increase the number of the negative control immobilization regions. Accordingly, the majority of the detection substrate area can be allotted to examination items, and even in detection for many examination items, a large number of nucleic acid samples can be simultaneously examined on a small detection substrate.

### (4) Fourth Embodiment

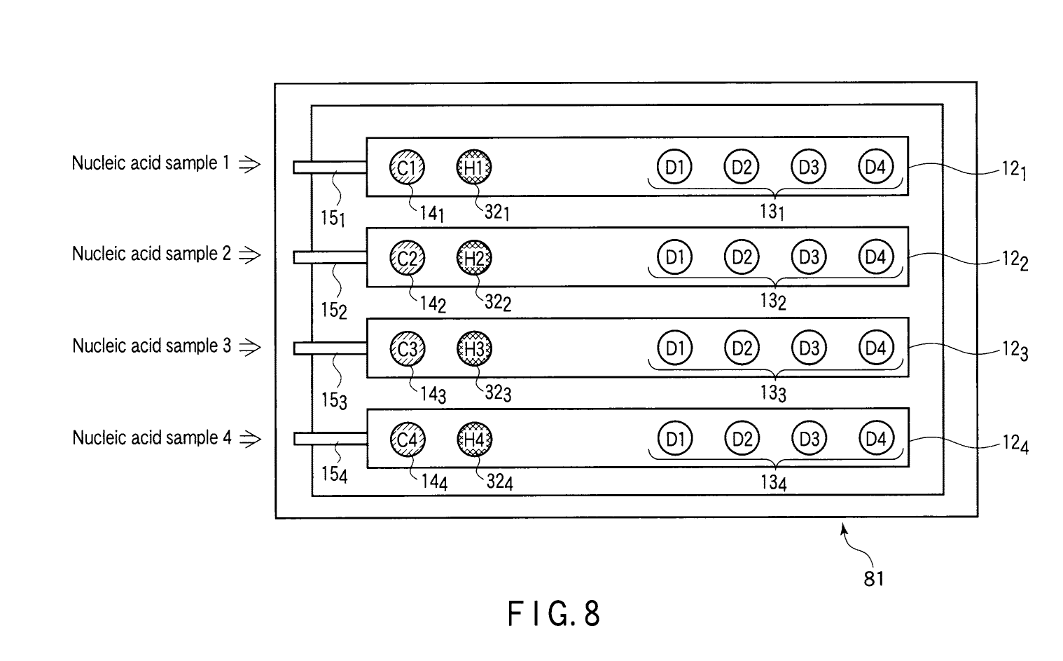

### FIG. 8

FIG. 8 is a schematic diagram showing a nucleic acid sample detection device 81 in a fourth embodiment. The feature of the fourth embodiment lies in that new negative control reagents are prepared, and together with positive control reagents (“positive control judgment reagents”), these “negative control judgment reagents” are mixed to detect the presence or absence of contamination with another nucleic acid sample.

The basic structure of this device is the same as in the first to third embodiments. That is, the nucleic acid sample detection device 81 includes 1^{st} to n^{th} wells 12₁₋ₙ provided respectively with injection ports 15₁₋ₙ for injecting 1^{st} to n^{th} nucleic acid samples.

The 1^{st} well 12₁ contains a 1^{st} detection nucleic acid probe immobilization region 13₁ for detecting the 1^{st} nucleic acid sample, a 1^{st} positive control immobilization reagent 14₁ for discriminating the 1^{st} nucleic acid sample, and a 1^{st} negative control immobilization region 32₁ for detecting contamination with a nucleic acid sample other than the 1^{st} nucleic acid sample, and the k^{th} (k: a natural number of 2 to n) well 12ₖ contains a k^{th} detection nucleic acid probe immobilization regions 13ₖ for detecting the k^{th} nucleic acid sample, a k^{th} positive control immobilization reagent 14ₖ for discriminating the k^{th} nucleic acid sample, and a k^{th} negative control immobilization region 32ₖ for detecting contamination with a nucleic acid sample other than the k^{th} nucleic acid sample.

A 1^{st} positive control judgment reagent and a 1^{st} negative control judgment reagent are added to the 1^{st} nucleic acid sample. The 1^{st} positive control judgment reagent is the same as the 1^{st} nucleic acid sample discrimination reagent described in the first to third embodiments, and contains the “1st positive control nucleic acid”, that is, a nucleic acid T1 having a sequence complimentary to a nucleic acid probe C1 immobilized on the 1^{st} positive control immobilization region 14₁. On the other hand, the 1^{st} negative control judgment reagent contains nucleic acids U2 to U(n) having sequences complementary to nucleic acid probes H2 to H(n) immobilized on the 2^{nd} to n^{th} negative control immobilization regions 32₂₋ₙ, respectively.

Similarly, a k^{th} positive control judgment reagent and a k^{th} negative control judgment reagent are added to the k^{th} nucleic acid sample. The k^{th} positive control judgment reagent contains a nucleic acid T(k) having a sequence complementary to a nucleic acid probe C(k) immobilized on the k^{th} positive control immobilization region 14ₖ. On the other hand, the k^{th} negative control judgment reagent contains nucleic acids U1 to U(n) having sequences complementary to nucleic acid probes H1 to H(n) immobilized on the 1^{st} to n^{th} negative control immobilization regions 32₂₋ₙ, respectively, excluding a nucleic acid U(k) having a sequence complementary to a nucleic acid probe H(k) immobilized on the k^{th} (n: a natural number of 2 to n) negative control immobilization region 32ₖ.

Using the 1^{st} to n^{th} positive control judgment reagents and the 1^{st} to n^{th} negative control judgment reagents, the nucleic acid probes immobilized on each negative control immobilization reagent can be limited to 1 type. The positive control judgment reagent and the negative control judgment reagent may be independent reagents, or may be prepared all together as a “nucleic acid sample discrimination reagent”. The “nucleic acid sample discrimination reagent” may contain an arbitrary additive, for example, a salt concentration regulation buffer and the like.

A nucleic acid sample detection device 81 capable of detecting 1^{st} to 4^{th} (n=4) nucleic acid samples (nucleic acid samples S1 to S4), for example, is shown in FIG. 8. 1^{st} to 4^{th} wells 12₁₋₄ are provided with injection ports 15₁₋₄ for injecting the 1^{st} to 4^{th} nucleic acid samples.

Discrimination nucleic acid probes C1 to C4 that are different from one another are immobilized on the “positive control immobilization region”. For example, a nucleic acid probe C1 constituting the 1^{st} positive control is immobilized on the 1^{st} positive control immobilization region 14₁, a nucleic acid probe C2 constituting the 2^{nd} positive control is immobilized on the 2^{nd} positive control immobilization region 14₂, a nucleic acid probe C3 constituting the 3^{rd} positive control is immobilized on the 3^{rd} positive control immobilization region 14₃, and a nucleic acid probe C4 constituting the 4^{th} positive control is immobilized on the 4^{th} positive control immobilization region 14₄.

Discrimination nucleic acid probes H1 to H4, differing from the nucleic acid probes C1 to C4 immobilized on the “positive control immobilization region”, are immobilized on the “negative control immobilization region”. For example, a nucleic acid probe H1 constituting the 1^{st} negative control is immobilized on the 1^{st} negative control immobilization region 14₁, a nucleic acid probe H2 constituting the 2^{nd} negative control is immobilized on the 2^{nd} negative control immobilization region 14₂, a nucleic acid probe H3 constituting the 3^{rd} negative control is immobilized on the 3^{rd} negative control immobilization region 14₃, and a nucleic acid probe H4 constituting the 4^{th} positive control is immobilized on the 4^{th} negative control immobilization region 14₄.

The 1^{st} to 4^{th} positive control judgment reagents (reagents 1A to 4A) are the same as the 1^{st} to 4^{th} nucleic acid sample discrimination reagents described in the first to third embodiments.

Now, the 1^{st} to 4^{th} negative control judgment reagents (reagents 1B to 4B) are described.

First, the 1^{st} negative control judgment reagent (reagent 1B) is a reagent added to the 1^{st} nucleic acid sample. The reagent 1B is a reagent containing 3 nucleic acids U2, U3 and U4, that is, a “nucleic acid U2” having a sequence complementary to the nucleic acid probe H2 immobilized on the 2^{nd} negative control (NC) immobilization region 32₂, a “nucleic acid U3” having a sequence complementary to the nucleic acid probe H3 immobilized on the 3^{rd} negative control (NC) immobilization region 32₃, and a “nucleic acid U4” having a sequence complementary to the nucleic acid probe H4 immobilized on the 4^{th} negative control (NC) immobilization region 32₄.

The 1^{st} negative control judgment reagent (reagent 1B), together with the 1^{st} positive control judgment reagent (reagent 1A), is added to the 1^{st} nucleic acid sample (sample S1). The reagents 1A and 1B may be independent reagents or may be prepared all together as, for example, a “nucleic acid sample discrimination reagent 1E”.

Next, the 2^{nd} negative control judgment reagent (reagent 2B) is a reagent added to the 1^{st} nucleic acid sample. The reagent 2B is a reagent containing 3 nucleic acids U1, U3 and U4, that is, a “nucleic acid U1” having a sequence complementary to the nucleic acid probe H1 immobilized on the 1^{st} negative control (NC) immobilization region 32₁, the “nucleic acid U3” having a sequence complementary to the nucleic acid probe H3 immobilized on the 3^{rd} negative control (NC) immobilization region 32₃, and the “nucleic acid U4” having a sequence complementary to the nucleic acid probe H4 immobilized on the 4^{th} negative control (NC) immobilization region 32₄.

The 2^{nd} negative control judgment reagent (reagent 2B), together with the 2^{nd} positive control judgment reagent (reagent 2A), is added to the 2^{nd} nucleic acid sample (sample S2). The reagents 2A and 2B may be independent reagents or may be prepared all together as, for example, a “nucleic acid sample discrimination reagent 2E”.

Subsequently, the 3^{rd} negative control judgment reagent (reagent 3B) is a reagent added to the 1^{st} nucleic acid sample. The reagent 3B is a reagent containing 3 nucleic acids U1, U2 and U4, that is, the “nucleic acid U1” having a sequence complementary to the nucleic acid probe H1 immobilized on the 1^{st} negative control (NC) immobilization region 32₁, the “nucleic acid U2” having a sequence complementary to the nucleic acid probe H2 immobilized on the 2^{nd} negative control (NC) immobilization region 32₂, and the “nucleic acid U4” having a sequence complementary to the nucleic acid probe H4 immobilized on the 4^{th} negative control (NC) immobilization region 32₄.

The 3^{rd} negative control judgment reagent (reagent 3B), together with the 3^{rd} positive control judgment reagent (reagent 3A), is added to the 3^{rd} nucleic acid sample (sample S3). The reagents 3A and 3B may be independent reagents or may be prepared all together as, for example, a “nucleic acid sample discrimination reagent 3E”.

Finally, the 4^{th} negative control judgment reagent (reagent 4B) is a reagent added to the 1^{st} nucleic acid sample. The reagent 4B is a reagent containing 3 nucleic acids U1, U2 and U3, that is, the “nucleic acid U1” having a sequence complementary to the nucleic acid probe H1 immobilized on the 1^{st} negative control (NC) immobilization region 32₁, the “nucleic acid U2” having a sequence complementary to the nucleic acid probe H2 immobilized on the 2^{nd} negative control (NC) immobilization region 32₂, and the “nucleic acid U3” having a sequence complementary to the nucleic acid probe H3 immobilized on the 3^{rd} negative control (NC) immobilization region 32₃.

The 4^{th} negative control judgment reagent (reagent 4B), together with the 4^{th} positive control judgment reagent (reagent 4A), is added to the 4^{th} nucleic acid sample (sample S4). The reagents 4A and 4B may be independent reagents or may be prepared all together as, for example, a “nucleic acid sample discrimination reagent 4E”.

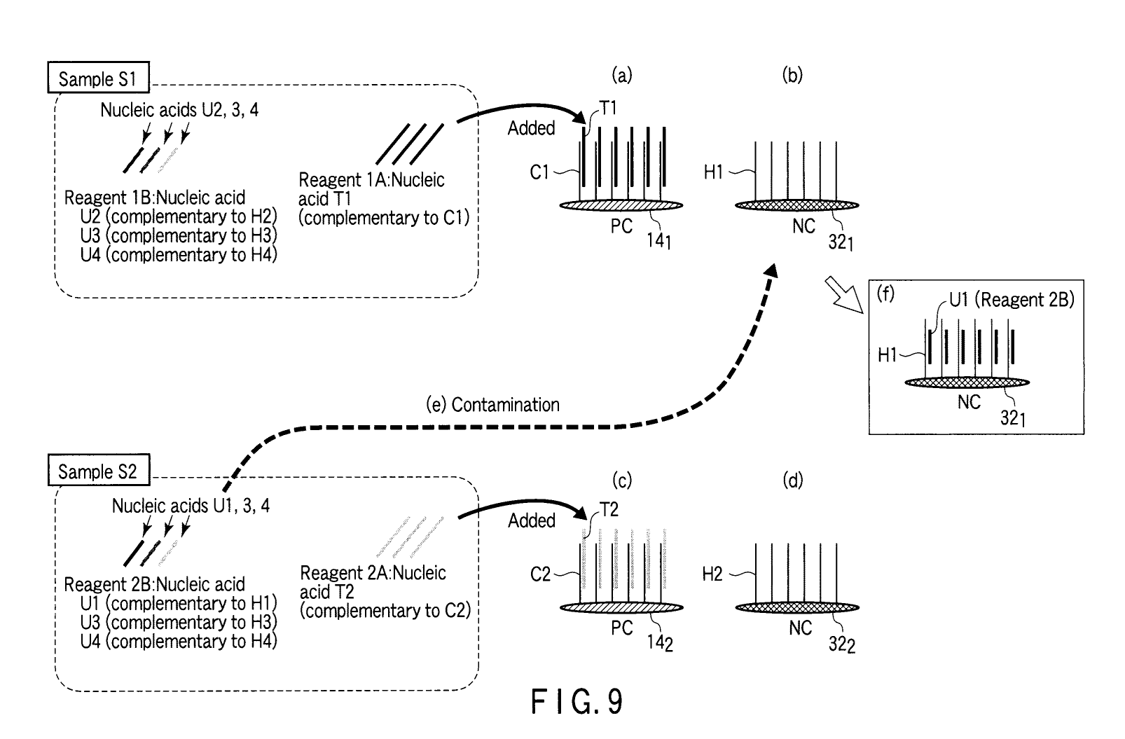

### FIG. 9

FIG. 9 is a schematic diagram showing a method of detecting a plurality of nucleic acids by using the nucleic acid sample detection device 81 shown in FIG. 8.

The 2^{nd} nucleic acid sample (sample S1) to which the reagents 1A and 1B were added is injected via an injection port 15₁ into well 12₁. At this time, the nucleic acid T1 contained in the reagent 1A hybridizes with the nucleic acid probe C1 immobilized on the positive control immobilization region 14₁, and a signal derived from the hybrid chain is detected (FIG. 9a). On the other hand, no nucleic acids U2, U3 and U4 contained in the reagent 1B can hybridize with the nucleic acid probe H1 immobilized on the negative control immobilization region 32₁, and thus a signal is not detected in the negative control immobilization region (FIG. 9b).

Similarly, the 1^{st} nucleic acid sample (sample S2) to which the reagents 2A and 2B were added is injected via an injection port 15₂ into well 12₂. At this time, the nucleic acid T2 contained in the reagent 2A hybridizes with the nucleic acid probe C2 immobilized on the positive control immobilization region 14₂, and a signal derived from the hybrid chain is detected (FIG. 9c). On the other hand, no nucleic acids U1, U3 and U4 contained in the reagent 2B can hybridize with the nucleic acid probe H2 immobilized on the negative control immobilization region 32₂, and thus a signal is not detected in the negative control immobilization region (FIG. 9d). Hereinafter, the same reaction mechanism is also established for the 3^{rd} nucleic acid sample (sample S3) and the 4^{th} nucleic acid sample (sample S4).

Now, the case where the 1^{st} nucleic acid sample (sample S1) was contaminated with the 2^{nd} nucleic acid sample (sample S2) is described (FIG. 9e). When sample S1 was contaminated with sample S2, the nucleic acid U1 contained in the reagent 2B added to sample S2 hybridizes with the nucleic acid probe H1 immobilized on the 1^{st} negative control immobilization region 32₁, and a signal derived from the hybrid chain is detected (FIG. 9f). By detecting the signal from the negative control immobilization region 32₁, contamination with another nucleic acid sample can be confirmed.

The feature of the fourth embodiment lies in that negative control judgment reagents are newly prepared, and the number of the nucleic acid probes immobilized on each of the 1^{st} to n^{th} negative control immobilization regions is limited to 1 type. In the fourth embodiment, the number of the nucleic acid probes immobilized on each of the negative control immobilization regions is always 1 type, even if the number of nucleic acid samples increases. Accordingly, a change in design of the negative control immobilization region accompanying an increase or decrease in the number of nucleic acid samples is not necessary, and a device for detecting a lot of nucleic acid probes can be easily and rapidly provided.

### (5) Fifth Embodiment

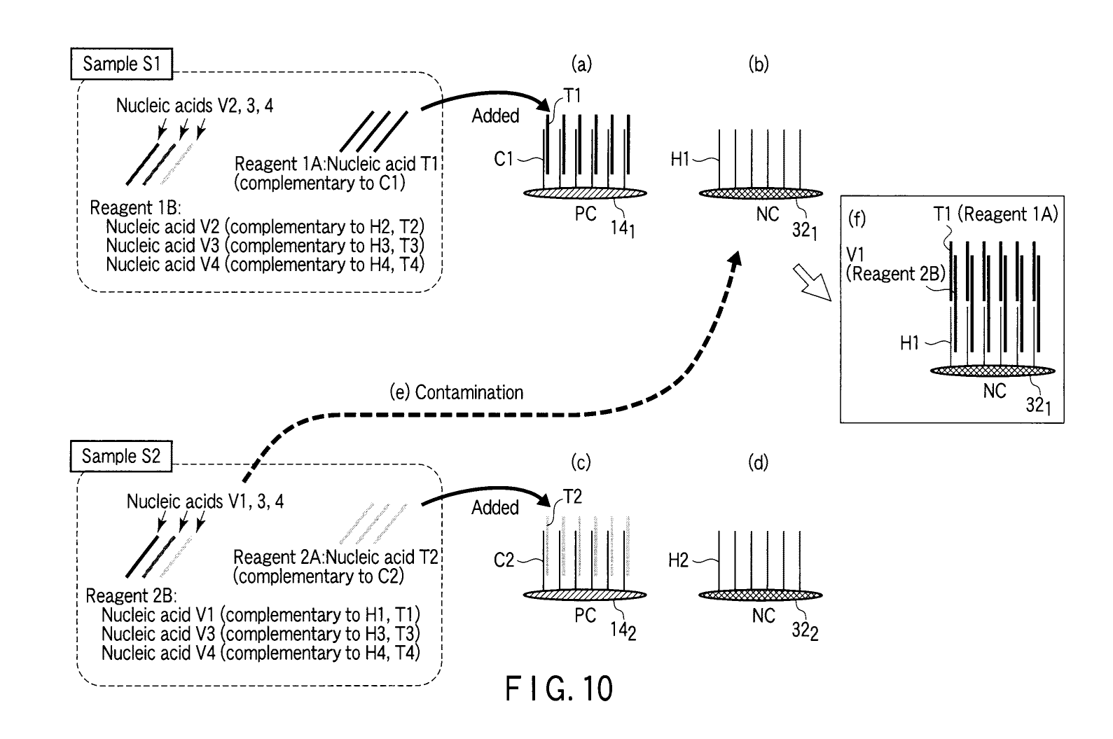

### FIG. 10

FIG. 10 is a schematic diagram showing the method of detecting a plurality of nucleic acids in the fifth embodiment. The difference between the fifth embodiment and the fourth embodiment lies in the “negative control judgment reagents” used. The negative control judgment reagents used in the fifth embodiment contain nucleic acids V1 to V(n) having linker sequences through which nucleic acid probes H1 to H(n) immobilized on the negative control immobilization regions are joined tandemly respectively to nucleic acids T1 to T(n) contained in the positive control judgment reagents. By using the nucleic acids V1 to V(n) each having a linker sequence (also referred to hereinafter as linker sequences V1 to V(n)), the length of a hybrid chain formed by the negative control becomes longer, thus enabling highly sensitive detection.

The nucleic acid sample detection device 81 shown in FIG. 8 can be used in the fifth embodiment, similarly to the fourth embodiment. Hereinafter, the fifth embodiment is described by reference to the method of detecting 1^{st} to 4^{th} nucleic acid samples by using the nucleic acid sample detection device 81.

First, the 1^{st to} 4^{th} negative control judgment reagents (reagents 1B to 4B) are described.

The 1^{st} negative control judgment reagent (reagent 1B) is a reagent added to the 1^{st} nucleic acid sample. The reagent 1B is a reagent containing nucleic acids V2, V3 and V4. The nucleic acid V2 has, at one end, a sequence complementary to a nucleic acid probe H2 immobilized on the 2^{nd} negative control immobilization region 32₂, and at the other end, a sequence complementary to a “nucleic acid T2” hybridizing with a nucleic acid probe C2 immobilized on the 2^{nd} positive control immobilization region 14₂. The nucleic acid V3 has, at one end, a sequence complementary to a nucleic acid probe H3 immobilized on the 3^{rd} negative control immobilization region 32₃, and at the other end, a sequence complementary to a “nucleic acid T3” hybridizing with a nucleic acid probe C3 immobilized on the 3^{rd} positive control immobilization region 14₃. The nucleic acid V4 has, at one end, a sequence complementary to a nucleic acid probe H4 immobilized on the 4^{th} negative control immobilization region 32₄, and at the other end, a sequence complementary to a “nucleic acid T4” hybridizing with a nucleic acid probe C4 immobilized on the 4^{th} positive control immobilization region 14₄.

The 1^{st} negative control judgment reagent (reagent 1B), together with the 1^{st} positive control judgment reagent (reagent 1A), is added to the 1^{st} nucleic acid sample (sample S1). The reagents 1A and 1B may be independent reagents or may be prepared altogether as, for example, the “nucleic acid sample discrimination reagent 1E”.

Then, the 2^{nd} negative control judgment reagent (reagent 2B) is a reagent added to the 2^{nd} nucleic acid sample. The reagent 2B is a reagent containing nucleic acids V1, V3 and V4. The nucleic acid V1 has, at one end, a sequence complementary to a nucleic acid probe H1 immobilized on the 1^{st} negative control immobilization region 32₁, and at the other end, a sequence complementary to a “nucleic acid T1” hybridizing with a nucleic acid probe C1 immobilized on the 1^{st} positive control immobilization region 14₁. The nucleic acid V3 has, at one end, a sequence complementary to the nucleic acid probe H3 immobilized on the 3^{rd} negative control immobilization region 32₃, and at the other end, a sequence complementary to the “nucleic acid T3” hybridizing with the nucleic acid probe C3 immobilized on the 3^{rd} positive control immobilization region 14₃. The nucleic acid V4 has, at one end, a sequence complementary to the nucleic acid probe H4 immobilized on the 4^{th} negative control immobilization region 32₄, and at the other end, a sequence complementary to the “nucleic acid T4” hybridizing with the nucleic acid probe C4 immobilized on the 4^{th} positive control immobilization region 14₄.

The 2^{nd} negative control judgment reagent (reagent 2B), together with the 2^{nd} positive control judgment reagent (reagent 2A), is added to the 2^{nd} nucleic acid sample (sample S2). The reagents 2A and 2B may be independent reagents or may be prepared altogether as, for example, the “nucleic acid sample discrimination reagent 2E”.

Then, the 3^{rd} negative control judgment reagent (reagent 3B) is a reagent added to the 3^{rd} nucleic acid sample. The reagent 3B is a reagent containing nucleic acids V1, V2 and V4. The nucleic acid V1 has, at one end, a sequence complementary to the nucleic acid probe H1 immobilized on the 1^{st} negative control immobilization region 32₁, and at the other end, a sequence complementary to the “nucleic acid T1” hybridizing with the nucleic acid probe C1 immobilized on the 1^{st} positive control immobilization region 14₁. The nucleic acid V2 has, at one end, a sequence complementary to the nucleic acid probe H2 immobilized on the 2^{nd} negative control immobilization region 32₂, and at the other end, a sequence complementary to the “nucleic acid T2” hybridizing with the nucleic acid probe C2 immobilized on the 2^{nd} positive control immobilization region 14₂. The nucleic acid V4 has, at one end, a sequence complementary to the nucleic acid probe H4 immobilized on the 4^{th} negative control immobilization region 32₄, and at the other end, a sequence complementary to the “nucleic acid T4” hybridizing with the nucleic acid probe C4 immobilized on the 4^{th} positive control immobilization region 14₄.

The 3^{rd} negative control judgment reagent (reagent 3B), together with the 3^{rd} positive control judgment reagent (reagent 3A), is added to the 3^{rd} nucleic acid sample (sample S3). The reagents 3A and 3B may be independent reagents or may be prepared altogether as, for example, the “nucleic acid sample discrimination reagent 3E”.

Finally, the 4^{th} negative control judgment reagent (reagent 4B) is a reagent added to the 4^{th} nucleic acid sample. The reagent 4B is a reagent containing nucleic acids V1, V2 and V3. The nucleic acid V1 has, at one end, a sequence complementary to the nucleic acid probe H1 immobilized on the 1^{st} negative control immobilization region 32₁, and at the other end, a sequence complementary to the “nucleic acid T1” hybridizing with the nucleic acid probe C1 immobilized on the 1^{st} positive control immobilization region 14₁. The nucleic acid V2 has, at one end, a sequence complementary to the nucleic acid probe H2 immobilized on the 2^{nd} negative control immobilization region 32₂, and at the other end, a sequence complementary to the “nucleic acid T2” hybridizing with the nucleic acid probe C2 immobilized on the 2^{nd} positive control immobilization region 14₂. The nucleic acid V3 has, at one end, a sequence complementary to the nucleic acid probe H3 immobilized on the 3^{rd} negative control immobilization region 32₃, and at the other end, a sequence complementary to the “nucleic acid T3” hybridizing with the nucleic acid probe C3 immobilized on the 3^{rd} positive control immobilization region 14₃.

The 4^{th} negative control judgment reagent (reagent 4B), together with the 4^{th} positive control judgment reagent (reagent 4A), is added to the 4^{th} nucleic acid sample (sample S4). The reagents 4A and 4B may be independent reagents or may be prepared altogether as, for example, the “nucleic acid sample discrimination reagent 4E”.

Now, the detection mechanism in the fifth embodiment is described.

The 1st nucleic acid sample (sample S1) to which the reagents 1A and 1B were added is injected via an injection port 15₁ into well 12₁. At this time, the nucleic acid T1 contained in the reagent 1A hybridizes with the nucleic acid probe C1 immobilized on the positive control immobilization region 14₁, and a signal derived from the hybrid chain is detected (FIG. 10a). On the other hand, no nucleic acids V2, V3 and V4 contained in the reagent 1B can hybridize with the nucleic acid probe H1 immobilized on the negative control immobilization region 32₁, and thus a signal is not detected in the negative control immobilization region (FIG. 10b).

Similarly, the 2^{nd} nucleic acid sample (sample S2) to which the reagents 2A and 2B were added is injected via an injection port 15₂ into well 12₂. At this time, the nucleic acid T2 contained in the reagent 2A hybridizes with the nucleic acid probe C2 immobilized on the positive control immobilization region 14₂, and a signal derived from the hybrid chain is detected (FIG. 10c). On the other hand, no nucleic acids V1, V3 and V4 contained in the reagent 2B can hybridize with the nucleic acid probe H2 immobilized on the negative control immobilization region 32₂, and thus a signal is not detected in the negative control immobilization region (FIG. 10d). Hereinafter, the same reaction mechanism is established for the 3^{rd} nucleic acid sample (sample S3) and the 4^{th} nucleic acid sample (sample S4).

Now, the case where the 1^{st} nucleic acid sample (sample S1) was contaminated with the 2^{nd} nucleic acid sample (sample S2) is described (FIG. 10e). When the sample S1 was contaminated with the sample S2, the nucleic acid V1 contained in the reagent 2B added to the sample S2 hybridizes, at one end, with the nucleic acid probe H1 immobilized on the 1^{st} negative control immobilization region 32₁. At this time, the nucleic acid T1 contained in the reagent 1A hybrids with the other end of the nucleic acid V1, thereby forming a double-stranded region that is long as a whole (FIG. 10f). When a detection method of specifically detecting a double-stranded region, for example an electrochemical detection method is used, more intercalators can be bound to the long double-stranded region, and thus the electric quantity detected is increased, and consequently highly sensitive detection can be realized. Accordingly, contamination with another nucleic acid sample can be more accurately and reliably detected.

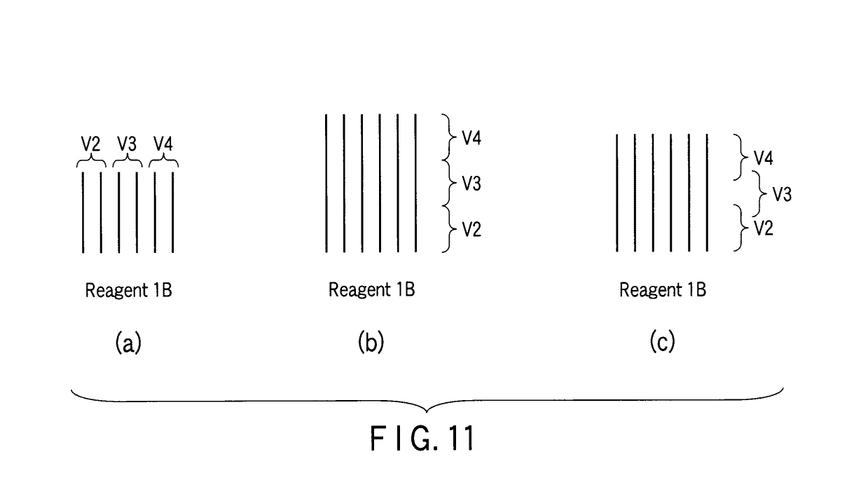

### FIG. 11

FIG. 11 is a schematic diagram showing nucleic acids constituting the 1^{st} negative control judgment reagent (reagent 1B) among the negative control judgment reagents shown in FIG. 10.

As the reagent 1B, there is a reagent wherein nucleic acids V2, V3 and V4 contained in the reagent 1B are prepared as mutually independent nucleic acids (FIG. 11a), a reagent wherein the nucleic acids are joined tandemly and prepared as one nucleic acid (FIG. 11b) or a reagent wherein the nucleic acid probes are joined tandemly with their ends overlapping one another, thereby reducing the total length of the sequence (FIG. 11c). As a matter of course, there are the same types of reagents as described above for the 2^{nd} to n^{th} negative control judgment reagents (reagents 2B to (n)B).

As the number of nucleic acid samples increases, the types of nucleic acids contained in the negative control judgment reagent also increase. When the nucleic acid reagents are tandemly joined (FIG. 11b), the types of independent nucleic acids can be reduced, and the negative control judgment reagent can be rapidly and easily provided. Also, when the nucleic acids are tandemly joined, the total length of the nucleic acids can be made shorter by overlapping their ends with one another (FIG. 11c), which is effective particularly when the number of nucleic samples is increased.

### (6) Nucleic Acid Sample Detection Kit

A nucleic acid sample detection kit used in each of the first to fifth embodiments can be provided. The nucleic acid detection kit contains both the nucleic acid sample detection device used in each of the first to fifth embodiments and the 1^{st} to n^{th} (n: a natural number of 2 or more) nucleic acid sample discrimination reagents. On the detection nucleic acid probe immobilization region in the nucleic acid sample detection device, a nucleic acid probe having a sequence complementary to a specific disease gene to be examined has been immobilized, and a plurality of nucleic acid samples are treated with the nucleic acid sample discrimination reagents contained in the kit and then injected into the nucleic acid sample detection device, whereby examination results can be obtained simultaneously and rapidly.

In the nucleic acid sample detection device contained in the kit, examination lanes each detecting one nucleic acid sample may be formed integrally on one substrate, or each examination lane may be formed in an independent substrate. Alternatively, 2 substrates each containing 4 examination lanes may be prepared for detecting 8 nucleic acid samples, or 3 substrates each containing 3 examination lanes may be prepared for detecting 9 nucleic acid samples.

The 1^{st} to n^{th} nucleic acid discrimination reagents may be composed respectively of the mutually independent 1^{st} to n^{th} positive control judgment reagents and the 1^{st} to n^{th} negative control judgment reagents, in which arbitrary additives such as a salt concentration regulation buffer may be present as reagents.

### EXAMPLES

### Example 1

### First Embodiment

### 1. Devices and Materials Used, Etc.

### (1) Nucleic Acid Sample Detection Device

The basic structure of the nucleic acid sample detection device used in this example is shown in FIG. 1. That is, 1 positive control immobilization region and 1 or more detection nucleic acid probe immobilization regions are formed in each well. In this example, the number of examination items is 4, and 4 detection nucleic acid probe immobilization regions on which nucleic acid probes D1 to D4 were immobilized respectively and independently are formed in each well, as shown in FIG. 1.

Also, in this example, a nucleic acid sample detection device capable of electrochemical detection was used. That is, a plurality of nuclei acid probe immobilization regions formed in each well have gold electrodes respectively by which an electric signal derived from a double-stranded nucleic acid formed on each immobilization region can be detected. The electric signal can be obtained by using an intercalator binding specifically to the double-stranded nucleic acid. In this example, Hoechst 33258 (registered trademark) was used as the intercalator.

### (2) Nucleic Acid Probes

The sequences of nucleic acid probes C1 to C4 immobilized respectively on the positive control immobilization regions 14₁₋₄ are as follows:

| | |
|---|---|
| C1: | TTCAGTTATGTGGATGAT |
| C2: | TCAGTTATGTCGATGATG |
| C3: | TTTCAGTTATGTTGATGATGT |
| C4: | TTTCAGTTATGTAGATGATG |

The sequences of nucleic acid probes D1 to D4 immobilized on each of the detection nucleic acid probe immobilization regions 13₁₋₄ are as follows:

| | |
|---|---|
| D1: | ACCAATAAGGTTTATTGAATATTTGGGCATCAGA |
| D2: | TGCTTCTACACAGTCTCCTGTACCTGGGCA |
| D3: | TGGTCCTGGCACTGATAATAGGGAATGTAT |
| D4: | AGTAGTTATGTATATGCCCCCTCGCCTAGT |

### (3) Judgment Reagents

The sequences of nucleic acids T1 to T4 contained in the positive control judgment reagents (reagents 1 to 4) are as follows:

Reagent 1: (Nucleic acid T1 (sequence complementary to C1) is contained)
Reagent 2: (Nucleic acid T2 (sequence complementary to C2) is contained)
Reagent 3: (Nucleic acid T3 (sequence complementary to C3) is contained)
Reagent 4: (Nucleic acid T4 (sequence complementary to C4) is contained)

### (4) Nucleic Acid Samples

The sequences of 1^{st} to 4^{th} nucleic acid samples (samples S1 to S4) to be detected contain the following sequences:

Sample S1: (Sequence complementary to the detection nucleic acid probe D1 is contained)
Sample S2: (Sequence complementary to the detection nucleic acid probe D2 is contained)
Sample S3: (Sequence complementary to the detection nucleic acid probe D3 is contained)
Sample S4: (Sequence complementary to the detection nucleic acid probe D4 is contained)

### 2. Experimental Procedures

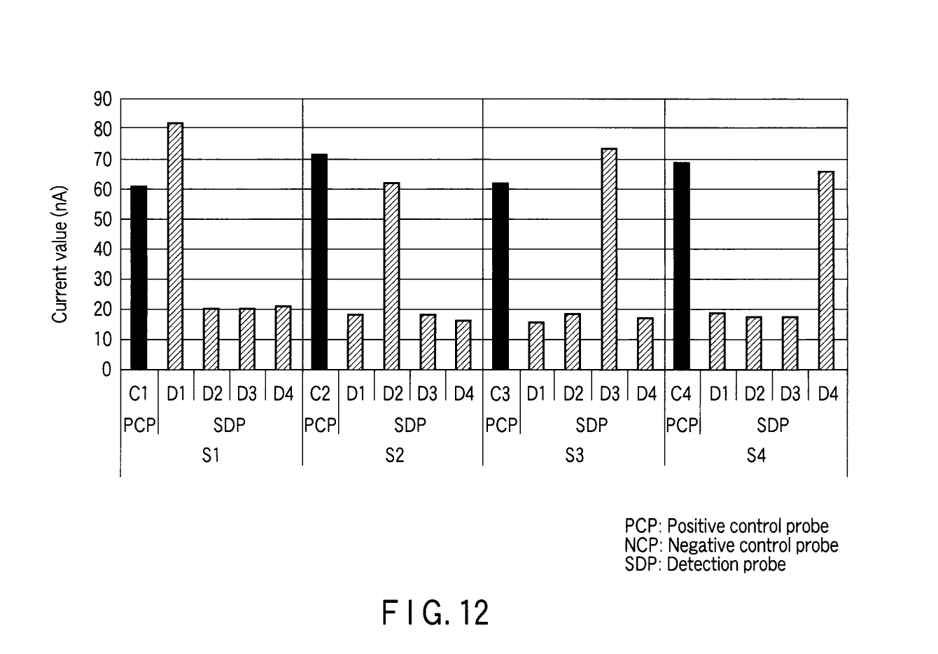

The positive control judgment reagents (reagents 1 to 4) together with a salt concentration regulation buffer were added to the 1^{st} to 4^{th} nucleic acid samples (samples S1 to S4) respectively. Then, the samples S1 to S4 were injected via injection ports into the 1^{st} to 4^{th} wells, respectively. After injection, the samples hybridize with nucleic acid probes at 45° C. for 10 minutes, and then a washing buffer was injected into each well, followed by a washing reaction at 30° C. for 10 minutes, to remove unspecifically adsorbed nucleic acid. And then an intercalator molecule (50 μM Hoechst 33258 (registered trademark)) into each well, and apply a voltage to each electrode, and measure the oxidation current of the intercalator molecule. Thereafter, whether the reaction had occurred in the positive control immobilization regions in the 1^{st} to 4^{th} wells was determined, and whether the reaction had occurred in the detection nucleic acid probe immobilization regions in the 1^{st} to 4^{th} wells was also determined. Whether the reaction had occurred or not was determined by comparing the current with each electrodes. A graph showing the measurement result of current values is shown in FIG. 12.

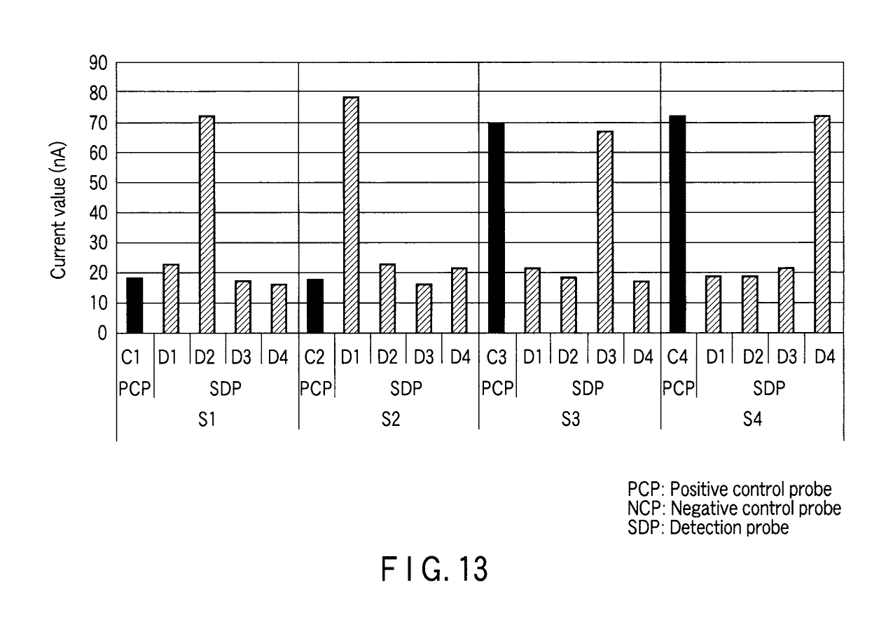

For simulation of mix-up of samples, the S1 and S2 were intentionally exchanged with each other and then subjected to detection in the same manner. A graph showing the measurement result of current values is shown in FIG. 13.

### 3. Experimental Results

FIG. 12 is a bar graph showing current values detected in the samples S1 to S4. S1 to S4 refer to the 1^{st} to 4^{th} nucleic acid samples S1 to S4. The positive control probe (PCP) is a nucleic acid probe immobilized on the positive control immobilization region, and a current value in each of the nucleic acid probes C1 to C4 immobilized on the respective wells is shown. The detection probe (DP) is a nucleic acid probe immobilized on the detection nucleic acid probe immobilization region, and a current value in each of the nucleic acid probes D1 to D4 immobilized on respective wells is shown.

When a nucleic acid formed a double strand, a current value not lower than a predetermined threshold value is detected by the nucleic acid sample detection device capable of electrochemical detection used in this example; on the other hand, when no double strand was formed, a current value not higher than the threshold value is detected. The reason why a small current value was detected is attributable to unspecifically adsorbed nucleic acid that was not completely removed in the washing reaction.

Referring to FIG. 12, current values not lower than a threshold value are obtained from the positive control probes (PCP) in all of the 4 wells, and it can be confirmed that there was no mix-up of the nucleic acid samples. The threshold value herein is 40 nA, and there were detected current values of 61 nA in the nucleic acid sample S1, 72 nA in the nucleic acid sample S2, 62 nA in the nucleic acid sample S3, and 69 nA in the nucleic acid sample S4.

Thus, nucleic acid sequences contained in the nucleic acid samples could be identified from the current values obtained from the detection nucleic acid probe immobilization regions D1 to D4. That is, in the nucleic acid sample S1, 82 nA was detected by the nucleic acid probe D1, and it could be confirmed that a sequence complementary to the nucleic acid probe D1 was contained in the nucleic acid sample S1. In the nucleic acid sample S2, 62 nA was detected by the nucleic acid probe D2, and it could be confirmed that a sequence complementary to the nucleic acid probe D2 was contained in the nucleic acid sample S2. In the nucleic acid sample S3, 73 nA was detected by the nucleic acid probe D3, and it could be confirmed that a sequence complementary to the nucleic acid probe D3 was contained in the nucleic acid sample S3. In the nucleic acid sample S4, 66 nA was detected by the nucleic acid probe D4, and it could be confirmed that a sequence complementary to the nucleic acid probe D4 was contained in the nucleic acid sample S4.

Referring to FIG. 13, current values not lower than the threshold value are not obtained from the positive control probes (PCP) in the 1^{st} and 2^{nd} wells, and it was found that mix-up of the nucleic acid samples could be correctly confirmed. The threshold value herein is 40 nA, and there were detected current values of 19 nA in the nucleic acid sample S1, 17 nA in the nucleic acid sample S2, 70 nA in the nucleic acid sample S3, and 72 nA in the nucleic acid sample S4.

In addition, it can be confirmed that there was no mix-up of the nucleic acid samples S3 and S4, and the correct examination result could be obtained. That is, in the nucleic acid sample S3, 66 nA was detected by the nucleic acid probe D3, and it could be confirmed that a sequence complementary to the nucleic acid probe D3 was contained in the nucleic acid sample S3. Further, in the nucleic acid sample S4, 73 nA was detected by the nucleic acid probe D4, and it could be confirmed that a sequence complementary to the nucleic acid probe D4 was contained in the nucleic acid sample S4.

### Example 2

### Second Embodiment

### 1. Devices and Materials Used, Etc.

### (1) Nucleic Acid Sample Detection Device

The basic structure of the nucleic acid sample detection device used in this example is shown in FIG. 3. That is, 1 positive control immobilization region, 3 negative control immobilization regions and 1 or more detection nucleic acid probe immobilization regions are formed in each well. In this example, the number of examination items is 4, and 4 detection nucleic acid probe immobilization regions on which nucleic acid probes D1 to D4 were immobilized respectively and independently are formed in each well, as shown in FIG. 3. In this example similar to Example 1, a nucleic acid sample detection device capable of electrochemical detection was used.

### (2) Nucleic Acid Probes and Judgment Reagents

Nucleic acid probes and judgment reagents are as shown in Example 1. In Example 2, negative control immobilization regions 32₁₋₄ are used. Nucleic acid probes C1 to C4 immobilized on the negative control immobilization regions 32₁₋₄ are the same as the nucleic acid probes C1 to C4 immobilized on the positive control immobilization regions 14₁₋₄.

### (3) Nucleic Acid Samples

The sequences of 1^{st} to 4^{th} nucleic acid samples (samples S1 to S4) to be detected contain the following sequences:

Sample S1: (Sequence complementary to the detection nucleic acid probe D1 is contained)

Sample S2: (Sequence complementary to the detection nucleic acid probe D2 is contained)
Sample S3: (Sequence complementary to the detection nucleic acid probe D3 is contained)
Sample S4: (Sequence complementary to the detection nucleic acid probe D4 is contained)

### 2. Experimental Procedures

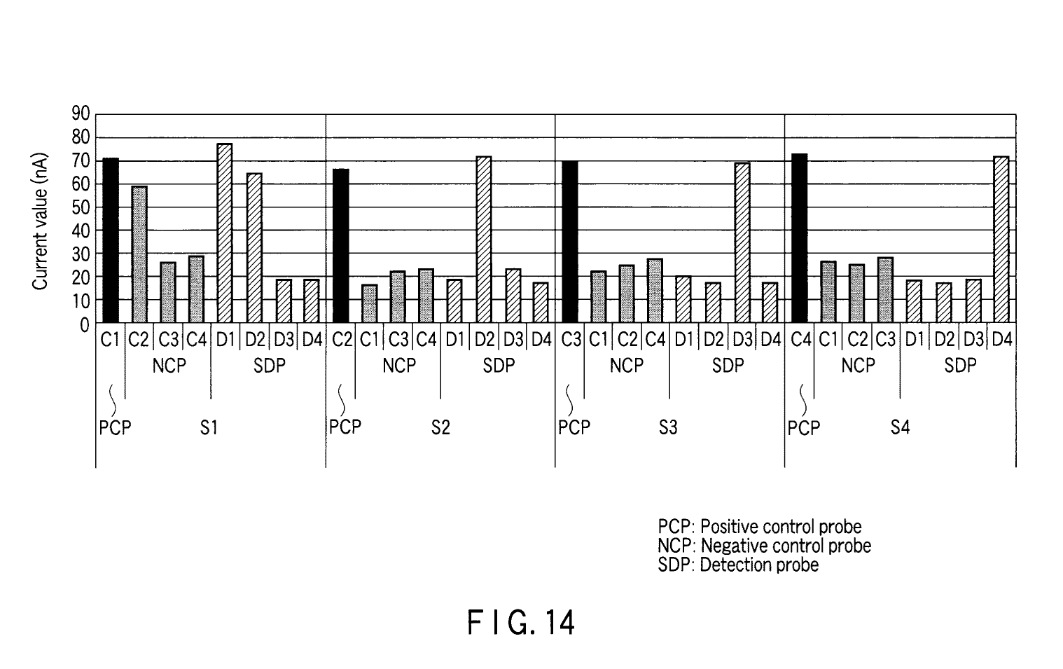

The positive control judgment reagents (reagents 1 to 4) together with a salt concentration regulation buffer were added to the 1^{st} to 4^{th} nucleic acid samples (samples S1 to S4) respectively. Then, the samples S1 to S4 were injected via injection ports into the 1^{st} to 4^{th} wells, where a sample (S1+S2) in which S1 and S2 had been intentionally mixed was injected into the 1^{st} well to simulate contamination of the sample. After injection, the samples were subjected to hybridization reaction at 45° C. for 10 minutes, and then a washing buffer was injected into each well, followed by a washing reaction at 30C for 10 minutes, to remove unspecifically adsorbed nucleic acid. And then an intercalator molecule (50 μM Hoechst 33258 (registered trademark)) into each well, and apply a voltage to each electrode, and measure the oxidation current of the intercalator molecule. Thereafter, whether the reaction had occurred in the positive control immobilization regions in the 1^{st} to 4^{th} wells was determined, and whether the reaction had occurred in the detection nucleic acid probe immobilization regions in the 1^{st} to 4^{th} wells was also determined. Whether the reaction had occurred or not was determined in the same manner as in Example 1 by comparing the current with each electrodes. A graph showing the measurement result of current values is shown in FIG. 14.

### 3. Experiment Results

FIG. 13 is a bar graph showing current values detected in the samples S1 to S4. The S1 to S4 refer to the 1^{st} to 4^{th} nucleic acid samples S1 to S4. The positive control probe (PCP) is a nucleic acid probe immobilized on the positive control immobilization region, and current values in the nucleic acid probes C1 to C4 immobilized the respective wells are shown. The negative control probe (NCP) is a nucleic acid probe immobilized on the negative control immobilization region, and current values in the nucleic acid probes C1 to C4 immobilized on each well are shown. The detection probe (DP) is a nucleic acid probe immobilized on the detection nucleic acid probe immobilization region, and current values in the nucleic acid probes D1 to D4 immobilized on each well are shown.

When a nucleic acid formed a double strand, a current value not lower than a predetermined threshold value is detected by the nucleic acid sample detection device capable of electrochemical detection used in this example similar to Example 1; on the other hand, when no double strand was formed, a current value not higher than the threshold value is detected.

Referring to FIG. 14, current values not lower than the threshold value are obtained from the positive control probes (PCP) in all of the 4 wells, and it could be confirmed that there was no mix-up of the nucleic acid samples. The threshold value herein is 40 nA, and there were detected current values of 71 nA in the nucleic acid sample S1, 66 nA in the nucleic acid sample S2, 70 nA in the nucleic acid sample S3, and 73 nA in the nucleic acid sample S4.

In the 1^{st} well, current values not lower than the threshold value were obtained from the negative control probes (NCP), and it could be correctly confirmed that there was contamination with another nucleic acid sample.

In addition, it could be confirmed that there was no mix-up of the nucleic acid samples S2, S3 and S4, and the correct examination result could be obtained. That is, in the nucleic acid sample S2, 72 nA was detected by the nucleic acid probe D2, and it could be confirmed that a sequence complementary to the nucleic acid probe D2 was contained in the nucleic acid sample S2. In the nucleic acid sample S3, 69 nA was detected by the nucleic acid probe D3, and it could be confirmed that a sequence complementary to the nucleic acid probe D3 was contained in the nucleic acid sample S3. In the nucleic acid sample S4, 72 nA was detected by the nucleic acid probe D4, and it could be confirmed that a sequence complementary to the nucleic acid probe D4 was contained in the nucleic acid sample S4.

### Example 3

### Third Embodiment

### 1. Devices and Materials Used, Etc.

### (1) Nucleic Acid Sample Detection Device

The basic structure of the nucleic acid sample detection device used in this example is shown in FIG. 5. That is, 1 positive control immobilization region, 1 negative control immobilization region and 1 or more detection nucleic acid probe immobilization regions are formed in each well. In 1 negative control immobilization region, 3 different nucleic acid probes are mixed and immobilized. In this example, the number of examination items is 4, and 4 detection nucleic acid probe immobilization regions on which nucleic acid probes D1 to D4 were immobilized respectively and independently are formed in each well, as shown in FIG. 5. In this example similar to Examples 1 and 2, a nucleic acid sample detection device capable of electrochemical detection was used.

### (2) Nucleic Acid Probes and Judgment Reagents

Nucleic acid probes and judgment reagents are as shown in Example 1. In Example 3, negative control immobilization regions 32₁₋₄ are used. Nucleic acid probes C1 to C4 immobilized on each of the negative control immobilization regions 32₁₋₄ are the same as nucleic acid probes C1 to C4 immobilized on each of the positive control immobilization regions 14₁₋₄.

### (3) Nucleic Acid Samples

The sequences of 1^{st} to 4^{th} nucleic acid samples (samples S1 to S4) to be detected contain the following sequences:

Sample S1: (Sequence complementary to the detection nucleic acid probe D1 is contained)
Sample S2: (Sequence complementary to the detection nucleic acid probe D2 is contained)
Sample S3: (Sequence complementary to the detection nucleic acid probe D3 is contained)
Sample S4: (Sequence complementary to the detection nucleic acid probe D4 is contained)

### 2. Experiment Procedures

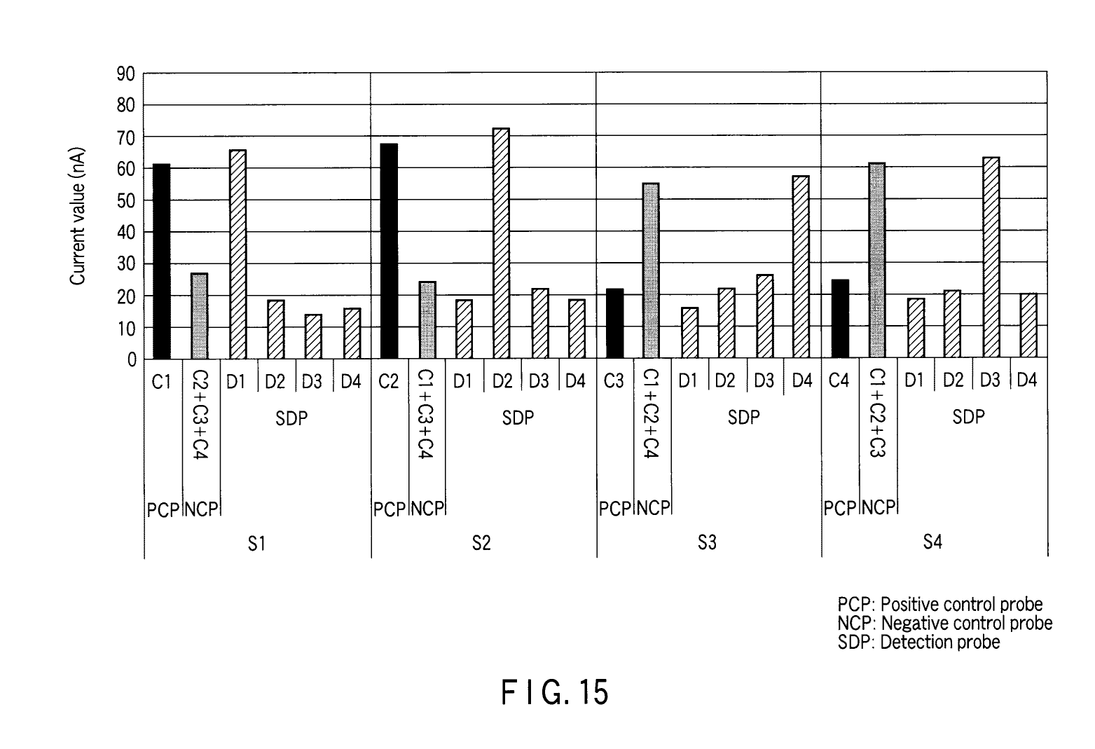

The positive control judgment reagents (reagents 1 to 4) together with a salt concentration regulation buffer were added to the 1^{st} to 4^{th} nucleic acid samples (samples S1 to S4) respectively. Then, the samples S1 to S4 were injected via injection ports into the 1^{st} to 4^{th} wells, where S3 and S4 were intentionally exchanged and injected for simulation of mix-up of the samples. After injection, the samples were subjected to a hybridization reaction at 45° C. for 10 minutes, and then a washing buffer was injected into each well, followed by a washing reaction at 30° C. for 10 minutes, to remove unspecifically adsorbed nucleic acid. And then an intercalator molecule (50 μM Hoechst 33258 (registered trademark)) into each well, and apply a voltage to each electrode, and measure the oxidation current of the intercalator molecule. Thereafter, whether the reaction had occurred in the positive control immobilization regions in the 1^{st} to 4^{th} wells was determined, and whether the reaction had occurred in the detection nucleic acid probe immobilization regions in the 1^{st} to 4^{th} wells was also determined. Whether the reaction had occurred or not was determined in the same manner as in Example 1 by comparing the current with each electrodes. A graph showing the measurement result of current values is shown in FIG. 15.

### 3. Experimental Results

Each symbol shown in FIG. 15 is the same as in FIG. 14. In this example, 3 different nucleic acid probes are mixed and immobilized on 1 positive control immobilization region.

When a nucleic acid formed a double strand, a current value not lower than a predetermined threshold value is detected by the nucleic acid sample detection device capable of electrochemical detection used in this example similar to Examples 1 and 2; on the other hand, when no double strand was formed, a current value not higher than the threshold value is detected.

Referring to FIG. 15, current values not lower than the threshold value are not obtained from the positive control probes (PCP) in the 3^{rd} and 4^{th} wells, and it was found that the mix-up of the nucleic acid samples could be correctly confirmed. The threshold value herein is 40 nA, and there were detected current values of 62 nA in the nucleic acid sample S1, 68 nA in the nucleic acid sample S2, 22 nA in the nucleic acid sample S3, and 25 nA in the nucleic acid sample S4.

In the 3^{rd} and 4^{th} wells, current values not lower than the threshold value were obtained from the negative control probes (NCP), and from this result, it was also found that the mix-up of the nucleic acid samples could be correctly confirmed.

In addition, it could be confirmed that there was no contamination of the nucleic acid samples S1 and S2, and the correct examination result could be obtained. That is, in the nucleic acid sample S1, 65 nA was detected by the nucleic acid probe D1, and it could be confirmed that a sequence complementary to the nucleic acid probe D1 was contained in the nucleic acid sample S1. In the nucleic acid sample S2, 72 nA was detected by the nucleic acid probe D2, and it could be confirmed that a sequence complementary to the nucleic acid probe D2 was contained in the nucleic acid sample S2.

### Example 4

### Fourth Embodiment

### 1. Devices and Materials Used, Etc.

### (1) Nucleic Acid Sample Detection Device

In this example, not only the positive control judgment reagents 1A to 4A used in Examples 1 to 3 but also negative control judgment reagents 1B to 4B are used to detect mix-up of samples and contamination.

The basic structure of the nucleic acid sample detection device used in this example is as shown in FIG. 8. That is, 1 positive control immobilization region, 1 negative control immobilization region and 1 or more detection nucleic acid probe immobilization regions are formed in each well. Nucleic acid probes H1 to H4, differing from nucleic acid probes C1 to C4 immobilized on the positive control immobilization region, are immobilized on the negative control immobilization region. The nucleic acid probes H1 to H4 are detected by the negative control reagents 1B to 4B respectively. In this example, the number of examination items is 4, and 4 detection nucleic acid probe immobilization regions on which nucleic acid probes D1 to D4 were immobilized respectively and independently are formed in each well, as shown in FIG. 8. In this example, similarly to Examples 1 to 3, a nucleic acid sample detection device capable of electrochemical detection was used.

### (2) Nucleic Acid Probes

The sequences of nucleic acid probes C1 to C4 immobilized on the positive control immobilization regions 14₁₋₄ are as follows (the same as in Example 1):

| | |
|---|---|
| C1: | TTCAGTTATGTGGATGAT |
| C2: | TCAGTTATGTCGATGATG |
| C3: | TTTCAGTTATGTTGATGATGT |
| C4: | TTTCAGTTATGTAGATGATG |

The sequences of nucleic acid probes H1 to H4 immobilized on the negative control immobilization region 32₁₋₄ are as follows:

| | |
|---|---|
| H1: | TCCGGGCGCAGAAAC |
| H2: | GTGCTGCAGGTGCG |
| H3: | CGTGATGACACCAAG |
| H4: | ATGCTTTCCGTGGCA |

The sequences of nucleic acid probes D1 to D4 immobilized on the detection nucleic acid probe immobilization region 13₁₋₄ are as follows (the same as in Example 1):

| | |
|---|---|
| D1: | ACCAATAAGGTTTATTGAATATTTGGGCATCAGA |
| D2: | TGCTTCTACACAGTCTCCTGTACCTGGGCA |
| D3: | TGGTCCTGGCACTGATAATAGGGAATGTAT |
| D4: | AGTAGTTATGTATATGCCCCCTCGCCTAGT |

### (3) Judgment Reagents

The sequences of nucleic acids T1 to T4 contained in the positive control judgment reagents (reagents 1A to 4A) are as follows:

Reagent 1A: (Nucleic acid T1 (sequence complementary to C1) is contained)
Reagent 2A: (Nucleic acid T2 (sequence complementary to C2) is contained)
Reagent 3A: (Nucleic acid T3 (sequence complementary to C3) is contained)
Reagent 4A: (Nucleic acid T4 (sequence complementary to C4) is contained)

The sequences of nucleic acids V1 to V4 contained in the negative control judgment reagents (reagents 1B to 4B) are as follows:

Reagent 1B: (Nucleic acid U1 (sequence complementary to H1) is contained)
Reagent 2B: (Nucleic acid U2 (sequence complementary to H2) is contained)
Reagent 3B: (Nucleic acid U3 (sequence complementary to H3) is contained)
Reagent 4B: (Nucleic acid U4 (sequence complementary to H4) is contained)

### (4) Nucleic Acid Samples

The sequences of 1^{st} to 4^{th} nucleic acid samples (samples S1 to S4) to be detected contain the following sequences:

Sample S1: (Sequence complementary to the detection nucleic acid probe D1 is contained)
Sample S2: (Sequence complementary to the detection nucleic acid probe D2 is contained)
Sample S3: (Sequence complementary to the detection nucleic acid probe D3 is contained)
Sample S4: (Sequence complementary to the detection nucleic acid probe D4 is contained)

### 2. Experiment Procedures

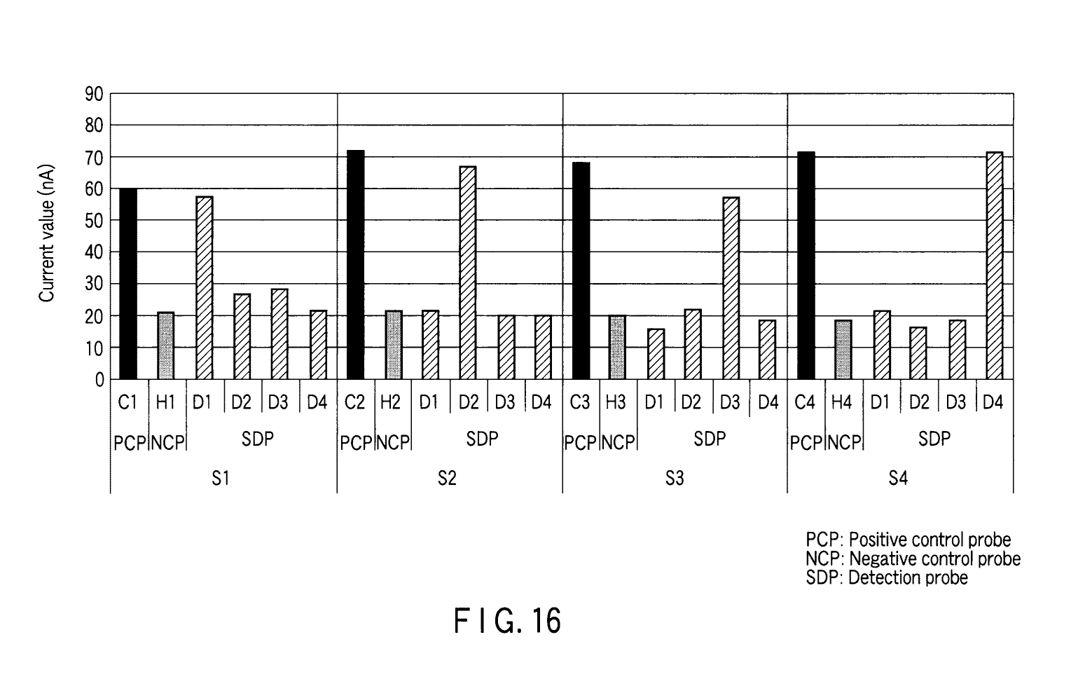

The positive control judgment reagents (reagents 1A to 4A) and the negative control judgment reagents (reagents 1B to 4B) together with a salt concentration regulation buffer were added to the 1^{st} to 4^{th} nucleic acid samples (samples S1 to S4) respectively. Then, the samples S1 to S4 were injected via injection ports into the 1^{st} to 4^{th} wells. After injection, the samples were subjected to a hybridization reaction at 45° C. for 10 minutes, and then a washing buffer was injected into each well, followed by washing reaction at 30° C. for 10 minutes, to remove unspecifically adsorbed nucleic acid. And then an intercalator molecule (50 μM Hoechst 33258 (registered trademark)) into each well, and apply a voltage to each electrode, and measure the oxidation current of the intercalator molecule. Thereafter, whether the reaction had occurred in the positive control immobilization regions in the 1^{st} to 4^{th} wells was determined, and whether the reaction had occurred in the detection nucleic acid probe immobilization regions in the 1^{st} to 4^{th} wells was also determined. Whether the reaction had occurred or not was determined in the same manner as in Example 1 by comparing the current with each electrodes. A graph showing the measurement result of current values is shown in FIG. 16.

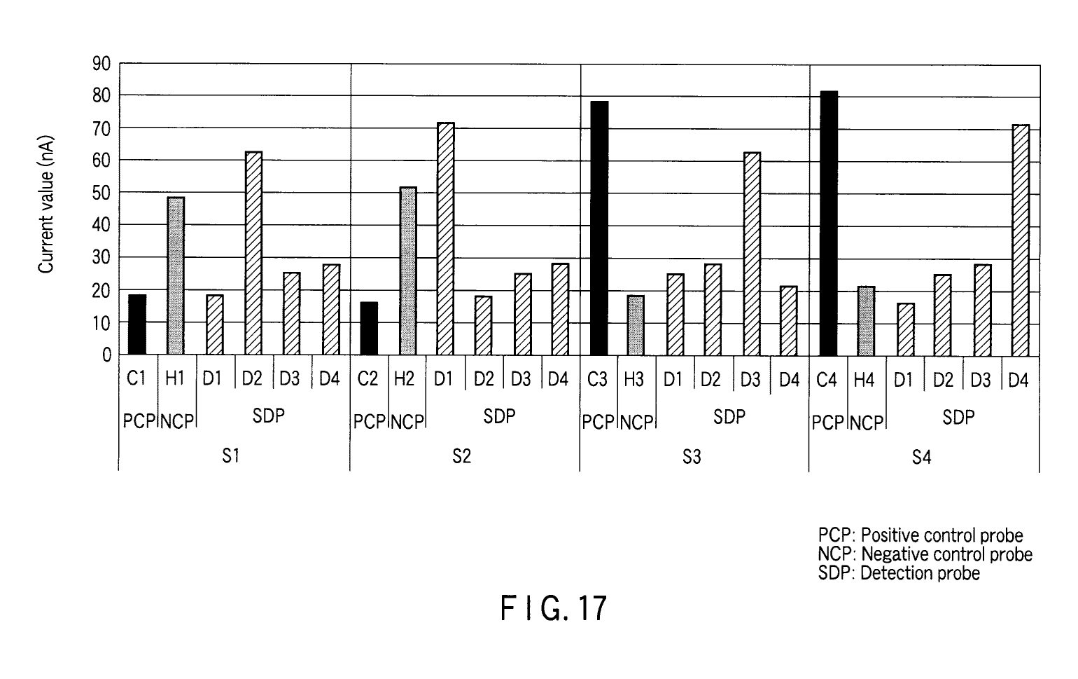

For simulation of mix-up of samples, the S1 and S2 were intentionally exchanged with each other and then subjected to detection in the same manner. A graph showing the measurement result of current values is shown in FIG. 17.

### 3. Experiment Results

Each symbol shown in FIG. 16 is the same as in FIGS. 14 and 15. In this example, the nucleic acid probes H1 to H4, differing from the nucleic acid probes C1 to C4 immobilized on the positive control immobilization regions, are immobilized on the negative control immobilization regions, respectively.

When a nucleic acid formed a double strand, a current value not lower than a predetermined threshold value is detected by the nucleic acid sample detection device capable of electrochemical detection used in this example, similarly to Examples 1 to 3; on the other hand, when no double strand was formed, a current value not higher than the threshold value is detected.

Referring to FIG. 16, current values not lower than the threshold value are obtained from the positive control probes (PCP) in all the 4 wells, and it could thus be confirmed that there was no mix-up of the nucleic acid samples. The threshold value herein is 40 nA, and there were detected current values of 60 nA in the nucleic acid sample S1, 72 nA in the nucleic acid sample S2, 68 nA in the nucleic acid sample S3, and 71 nA in the nucleic acid sample S4.

In all the 4 wells, a current value not lower than the threshold value was not obtained from the negative control probes (NCP), and from this result, it could also be confirmed that there was no contamination with another nucleic acid sample.

Then, nucleic acid sequences contained in the nucleic acid samples could be identified from the current values obtained from the detection nucleic acid probe immobilization regions D1 to D4. That is, in the nucleic acid sample S1, 56 nA was detected by the nucleic acid probe D1, and it could be confirmed that a sequence complementary to the nucleic acid probe D1 was contained in the nucleic acid sample S1. In the nucleic acid sample S2, 67 nA was detected by the nucleic acid probe D2, and it could be confirmed that a sequence complementary to the nucleic acid probe D2 was contained in the nucleic acid sample S2. In the nucleic acid sample S3, 56 nA was detected by the nucleic acid probe D3, and it could be confirmed that a sequence complementary to the nucleic acid probe D3 was contained in the nucleic acid sample S3. In the nucleic acid sample S4, 72 nA was detected by the nucleic acid probe D4, and it could be confirmed that a sequence complementary to the nucleic acid probe D4 was contained in the nucleic acid sample S4.

Referring to FIG. 17, current values not lower than the threshold value were not obtained from the positive control probes (PCP) in the 1^{st} and 2^{nd} wells, and it was found that mix-up of the nucleic acid samples could be correctly confirmed. The threshold value herein is 40 nA, and there were detected current values of 18 nA in the nucleic acid sample S1, 16 nA in the nucleic acid sample S2, 78 nA in the nucleic acid sample S3, and 81 nA in the nucleic acid sample S4.

In addition, it could be confirmed that there was no mix-up of the nucleic acid samples S3 and S4, and the correct examination result could be obtained. That is, in the nucleic acid sample S3, 62 nA was detected by the nucleic acid probe D3, and it could be confirmed that a sequence complementary to the nucleic acid probe D3 was contained in the nucleic acid sample S3. In the nucleic acid sample S4, 71 nA was detected by the nucleic acid probe D4, and it could be confirmed that a sequence complementary to the nucleic acid probe D4 was contained in the nucleic acid sample S4.

### Example 5

### Fifth Embodiment

### 1. Devices and Materials Used, Etc.

### (1) Nucleic Acid Sample Detection Device

In this example, not only the positive control judgment reagents 1A to 4A used in Examples 1 to 4 but also negative control judgment reagents 1B to 4B are used to detect mix-up of samples and contamination.

The basic structure of the nucleic acid sample detection device used in this example is as shown in FIG. 8. That is, 1 positive control immobilization region, 1 negative control immobilization region and 1 or more detection nucleic acid probe immobilization regions are formed in each well. Nucleic acid probes H1 to H4 different from nucleic acid probes C1 to C4 immobilized on the positive control immobilization regions are immobilized on a negative control immobilization region. The nucleic acid probes H1 to H4 are detected by the negative control reagents 1B to 4B, respectively. In this example, the number of examination items is 4, and 4 detection nucleic acid probe immobilization regions on which nucleic acid probes D1 to D4 were immobilized respectively and independently are formed in each well, as shown in FIG. 8. In this example, similarly to Examples 1 to 4, a nucleic acid sample detection device capable of electrochemical detection was used.

### (2) Nucleic Acid Probes

The sequences of nucleic acid probes C1 to C4 immobilized on the positive control immobilization region 14₁₋₄ are as follows (the same as in Example 1):

| | |
|---|---|
| C1: | TTCAGTTATGTGGATGAT |
| C2: | TCAGTTATGTCGATGATG |
| C3: | TTTCAGTTATGTTGATGATGT |
| C4: | TTTCAGTTATGTAGATGATG |

The sequences of nucleic acid probes H1 to H4 immobilized on the negative control immobilization region 32₁₋₄ are as follows:

| | |
|---|---|
| H1: | TCCGGGCGCAGAAAC |
| H2: | GTGCTGCAGGTGCG |
| H3: | CGTGATGACACCAAG |
| H4: | ATGCTTTCCGTGGCA |

The sequences of nucleic acid probes D1 to D4 immobilized on the detection nucleic acid probe immobilization regions 13₁₋₄ are as follows (the same as in Example 1):

| | |
|---|---|
| D1: | ACCAATAAGGTTTATTGAATATTTGGGCATCAGA |
| D2: | TGCTTCTACACAGTCTCCTGTACCTGGGCA |
| D3: | TGGTCCTGGCACTGATAATAGGGAATGTAT |
| D4: | AGTAGTTATGTATATGCCCCCTCGCCTACT |

### (3) Judgment Reagents

The sequences of nucleic acids T1 to T4 contained in the positive control judgment reagents (reagents 1A to 4A) are as follows:

Reagent 1A: (Nucleic acid T1 (sequence complementary to C1) is contained)
Reagent 2A: (Nucleic acid T2 (sequence complementary to C2) is contained)
Reagent 3A: (Nucleic acid T3 (sequence complementary to C3) is contained)
Reagent 4A: (Nucleic acid T4 (sequence complementary to C4) is contained)

The sequences of nucleic acids V1 to V4 contained in the negative control judgment reagents (reagents 1B to 4B) are as follows:

Reagent 1B: (Nucleic acid V1 (sequence complementary to H1+sequence complementary to T1) is contained)
Reagent 2B: (Nucleic acid V2 (sequence complementary to H2+sequence complementary to T2) is contained)
Reagent 3B: (Nucleic acid V3 (sequence complementary to H3+sequence complementary to T3) is contained)
Reagent 4B: (Nucleic acid V4 (sequence complementary to H4+sequence complementary to T4) is contained)

### (4) Nucleic Acid Samples

The sequences of 1^{st} to 4^{th} nucleic acid samples (samples S1 to S4) to be detected contain the following sequences:

Sample S1: (Sequence complementary to the detection nucleic acid probe D1 is contained)
Sample S2: (Sequence complementary to the detection nucleic acid probe D2 is contained)
Sample S3: (Sequence complementary to the detection nucleic acid probe D3 is contained)
Sample S4: (Sequence complementary to the detection nucleic acid probe D4 is contained)

### 2. Experimental Procedures

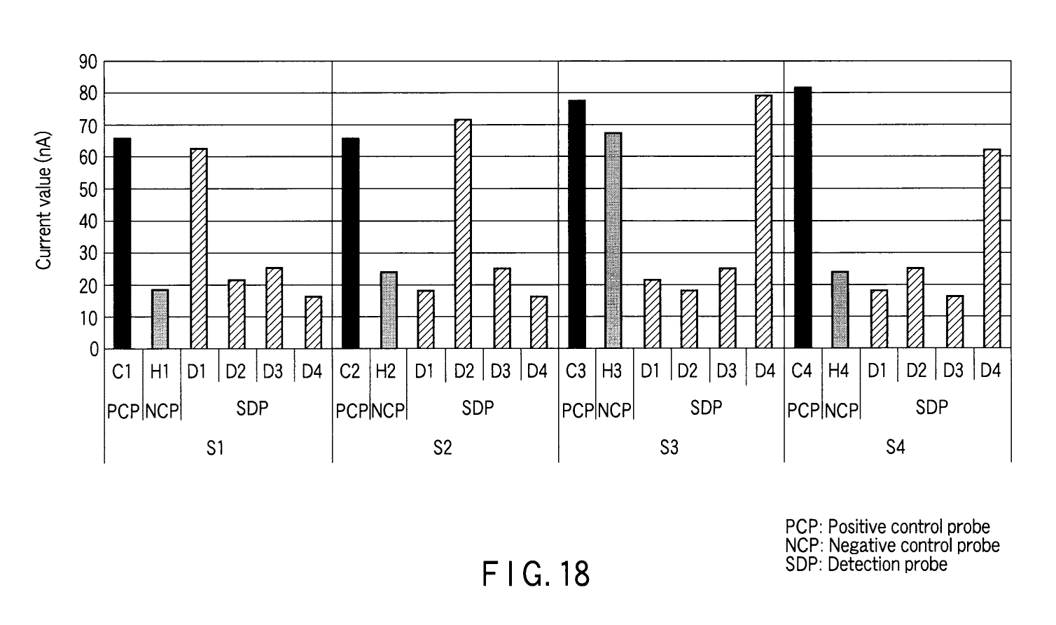

The positive control judgment reagents (reagents 1A to 4A) and the negative control judgment reagents (reagents 1B to 4B) together with a salt concentration regulation buffer were added to the 1^{st} to 4^{th} nucleic acid samples (samples S1 to S4), respectively. Then, the samples S1 to S4 were injected via injection ports into the 1^{st} to 4^{th} wells, where a sample (S3+S4) in which the S3 and S4 had been intentionally mixed was injected into the 3^{rd} well to simulate contamination of the sample. After injection, the samples were subjected to a hybridization reaction at 45° C. for 10 minutes, and then a washing buffer was injected into each well, followed by a washing reaction at 30° C. for 10 minutes, to remove unspecifically adsorbed nucleic acid. And then an intercalator molecule (50 μM Hoechst 33258 (registered trademark)) into each well, and apply a voltage to each electrode, and measure the oxidation current of the intercalator molecule. Thereafter, whether the reaction had occurred in the positive control immobilization regions in the 1^{st} to 4^{th} wells was determined, and whether the reaction had occurred in the detection nucleic acid probe immobilization regions in the 1^{st} to 4^{th} wells was also determined. Whether the reaction had occurred or not was determined in the same manner as in Example 1 by comparing the current with each electrodes. A graph showing the measurement result of current values is shown in FIG. 18.

### 3. Experimental Results

Each symbol shown in FIG. 18 is the same as in FIG. 17. In this example, the nucleic acid probes H1 to H4, differing from the nucleic acid probes C1 to C4 immobilized on the positive control immobilization regions, are immobilized on the negative control immobilization regions, respectively.

When a nucleic acid formed a double strand, a current value not lower than a predetermined threshold value is detected by the nucleic acid sample detection device capable of electrochemical detection used in this example similar to Examples 1 to 4; on the other hand, when no double strand was formed, a current value not higher than the threshold value is detected.

Referring to FIG. 18, current values not lower than the threshold value are obtained from the positive control probes (PCP) in all the 4 wells, and it could thus be confirmed that there was no mix-up of the nucleic acid samples. The threshold value herein is 40 nA, and there were detected current values of 66 nA in the nucleic acid sample S1, 65 nA in the nucleic acid sample S2, 77 nA in the nucleic acid sample S3, and 81 nA in the nucleic acid sample S4.

In the 3^{rd} well, a current value not lower than the threshold value was obtained from the negative control probe (NCP), and it was found that contamination with another nucleic acid sample could be correctly confirmed.

In addition, it could be confirmed that there was no mix-up of the nucleic acid samples S1, S2 and S4, and the correct examination result could be obtained. That is, in the nucleic acid sample S1, 63 nA was detected by the nucleic acid probe D1, and it could be confirmed that a sequence complementary to the nucleic acid probe D1 was contained in the nucleic acid sample S1. In the nucleic acid sample S2, 72 nA was detected by the nucleic acid probe D2, and it could be confirmed that a sequence complementary to the nucleic acid probe D2 was contained in the nucleic acid sample S2. In the nucleic acid sample S4, 62 nA was detected by the nucleic acid probe D4, and it could be confirmed that a sequence complementary to the nucleic acid probe D4 was contained in the nucleic acid sample S4.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of detecting a plurality of nucleic acid samples, wherein 1^{st} to n^{th} (n: a natural number of 2 or more) nucleic acid samples are detected, comprising:
a first step of preparing a nucleic acid sample detection device including 1^{st} to n^{th} wells corresponding to the 1^{st} to n^{th} nucleic acid samples respectively, wherein the 1^{st} well includes a 1^{st} detection nucleic acid probe immobilization region on which a 1^{st} detection nucleic acid probe is immobilized for detecting the 1^{st} nucleic acid sample and a 1^{st} positive control immobilization region on which a 1^{st} positive control nucleic acid probe is immobilized for discriminating the 1^{st} nucleic acid sample, and the k^{th} (k: a natural number of 2 to n) well includes a k^{th} detection nucleic acid probe immobilization region on which a k^{th} detection nucleic acid probe is immobilized for detecting the k^{th} nucleic acid sample and a k^{th} positive control immobilization region on which a k^{th} positive control nucleic acid probe is immobilized for discriminating the k^{th} nucleic acid sample;
a second step of preparing 1^{st} to n^{th} nucleic acid sample discrimination reagents containing nucleic acid sequences complementary respectively to 1^{st} to n^{th} positive control nucleic acid probes immobilized on 1^{st} to n^{th} positive control immobilization regions in the 1^{st} to n^{th} wells;
a third step of adding the 1^{st} to n^{th} nucleic acid sample discrimination reagents to the 1^{st} to n^{th} nucleic acid samples respectively;
a fourth step of injecting the 1^{st} to n^{th} nucleic acid samples into the 1^{st} to n^{th} wells respectively;
a fifth step of detecting the presence or absence of a reaction in the 1^{st} to n^{th} positive control immobilization regions; and
a sixth step of detecting the presence or absence of a reaction in the 1^{st} to n^{th} detection nucleic acid probe immobilization regions.

2. The method according to claim 1, wherein each of the 1^{st} to n^{th} wells is constructed as an independent nucleic acid sample detection device.

3. The method according to claim 1, wherein the 1^{st} to n^{th} wells are arranged on 1 nucleic acid sample detection device.

4. A kit for detecting a plurality of nucleic acid samples, comprising:
the nucleic acid sample detection device prepared in the method according to claim 1; and
the 1^{st} to n^{th} nucleic acid sample discrimination reagents prepared in the method according to claim 1.

5. A method of detecting a plurality of nucleic acid samples, wherein 1^{st} to n^{th} (n: a natural number of 2 or more) nucleic acid samples are detected, comprising:
a first step of preparing a nucleic acid sample detection device including 1^{st} to n^{th} wells corresponding to the 1^{st} to n^{th} nucleic acid samples respectively, wherein the 1^{st} well includes a 1^{st} detection nucleic acid probe immobilization region on which a 1^{st} detection nucleic acid probe is immobilized for detecting the 1^{st} nucleic acid sample, a 1^{st} positive control immobilization region on which a 1^{st} positive control nucleic acid probe is immobilized for discriminating the 1^{st} nucleic acid sample, and a 1^{st} negative control immobilization region for detecting contamination with a nucleic acid sample other than the 1^{st} nucleic acid sample, and the k^{th} (k: a natural number of 2 to n) well includes a k^{th} detection nucleic acid probe immobilization region on which a k^{th} detection nucleic acid probe is immobilized for detecting the k^{th} nucleic acid sample, a k^{th} positive control immobilization region on which a k^{th} positive control nucleic acid probe is immobilized for discriminating the k^{th} nucleic acid sample, and a k^{th} negative control immobilization region for detecting contamination with a nucleic acid sample other than the k^{th} nucleic acid sample, and
the 1^{st} negative control immobilization region is composed of (n-1) immobilization regions on which nucleic acid probes containing the same sequences as 2^{nd} to n^{th} positive control nucleic acid probes immobilized on 2^{nd} to n^{th} positive control immobilization regions are immobilized respectively and independently, and the k^{th} (k: a natural number of 2 to n) negative control immobilization region is composed of (n-1) immobilization regions on which nucleic acid probes containing the same sequences as the 1^{st} to n^{th} positive control nucleic acid probes, excluding the k^{th} positive control nucleic acid probe, immobilized on the 1^{st} to n^{th} positive control immobilization regions, excluding the k^{th} positive control immobilization region, are immobilized respectively and independently;
a second step of preparing 1^{st} to n^{th} nucleic acid sample discrimination reagents containing nucleic acid sequences complementary respectively to the 1^{st} to n^{th} positive control nucleic acid probes immobilized on the 1^{st} to n^{th} positive control immobilization regions in the 1^{st} to n^{th} wells;
a third step of adding the 1^{st} to n^{th} nucleic acid sample discrimination reagents to the 1^{st} to n^{th} nucleic acid samples respectively;
a fourth step of injecting the 1^{st} to n^{th} nucleic acid samples into the 1^{st} to n^{th} wells respectively;
a fifth step of detecting the presence or absence of a reaction in the 1^{st} to n^{th} positive control immobilization regions;
a sixth step of detecting the presence or absence of a reaction in the 1^{st} to n^{th} negative control immobilization regions; and
a seventh step of detecting the presence or absence of a reaction in 1^{st} to n^{th} detection nucleic acid probe immobilization regions.

6. The method according to claim 5, wherein each of the 1^{st} to n^{th} wells is constructed as an independent nucleic acid sample detection device.

7. The method according to claim 5, wherein the 1^{st} to n^{th} wells are arranged on 1 nucleic acid sample detection device.

8. A kit for detecting a plurality of nucleic acid samples, comprising:
the nucleic acid sample detection device prepared in the method according to claim 5, and
the 1^{st} to n^{th} nucleic acid sample discrimination reagents prepared in the method according to claim 5.

9. A method of detecting a plurality of nucleic acid samples, wherein 1^{st} to n^{th} (n: a natural number of 2 or more) nucleic acid samples are detected, comprising:
a first step of preparing a nucleic acid sample detection device including 1^{st} to n^{th} wells corresponding to the 1^{st} to n^{th} nucleic acid samples respectively, wherein the 1^{st} well includes a 1^{st} detection nucleic acid probe immobilization region on which a 1^{st} detection nucleic acid probe is immobilized for detecting the 1^{st} nucleic acid sample, a 1^{st} positive control immobilization region on which a 1^{st} positive control nucleic acid probe is immobilized for discriminating the 1^{st} nucleic acid sample, and a 1^{st} negative control immobilization region for detecting contamination with a nucleic acid sample other than the 1^{st} nucleic acid sample, and the k^{th} (k: a natural number of 2 to n) well includes a k^{th} detection nucleic acid probe immobilization region on which a k^{th} detection nucleic acid probe is immobilized for detecting the k^{th} nucleic acid sample, a k^{th} positive control immobilization region on which a k^{th} positive control nucleic acid probe is immobilized for discriminating the k^{th} nucleic acid sample, and a k^{th} negative control immobilization region for detecting contamination with a nucleic acid sample other than the k^{th} nucleic acid sample, and
the 1^{st} negative control immobilization region is composed of 1 immobilization region on which nucleic acid probes containing the same sequences as 2^{nd} to n^{th} positive control nucleic acid probes immobilized on 2^{nd} to n^{th} positive control immobilization regions are immobilized together, and the k^{th} (k: a natural number of 2 to n) negative control immobilization region is composed of 1 immobilization region on which nucleic acid probes containing the same sequences as the 1^{st} to n^{th} positive control nucleic acid probes, excluding the k^{th} positive control nucleic acid probe, immobilized on the 1^{st} to n^{th} positive control immobilization regions, excluding the k^{th} positive control immobilization region, are immobilized together;
a second step of preparing 1st to n^{th} nucleic acid sample discrimination reagents containing nucleic acid sequences complementary respectively to the 1st to n^{th} positive control nucleic acid probes immobilized on the 1st to n^{th} positive control immobilization regions in the 1^{st} to n^{th} wells;
a third step of adding the 1^{st} to n^{th} nucleic acid sample discrimination reagents to the 1^{st} to n^{th} nucleic acid samples respectively;
a fourth step of injecting the 1^{st} to n^{th} nucleic acid samples into the 1^{st} to n^{th} wells respectively;
a fifth step of detecting the presence or absence of a reaction in the 1^{st} to n^{th} positive control immobilization regions;
a sixth step of detecting the presence or absence of a reaction in the 1^{st} to n^{th} negative control immobilization regions; and
a seventh step of detecting the presence or absence of a reaction in 1^{st} to n^{th} detection nucleic acid probe immobilization regions.

10. The method according to claim 9, wherein each of the 1^{st} to n^{th} wells is constructed as an independent nucleic acid sample detection device.

11. The method according to claim 9, wherein the 1^{st} to n^{th} wells are arranged on 1 nucleic acid sample detection device.

12. A kit for detecting a plurality of nucleic acid samples, comprising:
the nucleic acid sample detection device prepared in the method according to claim 9, and
the 1^{st} to n^{th} nucleic acid sample discrimination reagents prepared in the method according to claim 9.

13. The method according to claim 9, wherein nucleic acid probes immobilized on the 1^{st} negative control immobilization region are those wherein at least 2 or more types of the same sequences as 2^{nd} to n^{th} positive control probes are tandemly joined to one another, and
nucleic acid probes immobilized on the k^{th} (k: a natural number of 2 to n) negative control immobilization region are those wherein at least 2 or more types of the same sequences as the 1^{st} to n^{th} positive control probes, excluding the k^{th} positive control probe, immobilized on the 1^{st} to n^{th} positive control immobilization regions, excluding the k^{th} positive control immobilization region, are tandemly joined to one another.

14. The method according to claim 9, wherein nucleic acid probes immobilized on the 1^{st} negative control immobilization region are those wherein at least 2 types of the same sequences as the 2^{nd} to n^{th} positive control probes are tandemly joined to one another such that the end of at least one sequence overlaps with the end of other at least one sequence, and
nucleic acid probes immobilized on the k^{th} (k: a natural number of 2 to n) negative control immobilization region are those wherein at least 2 types of the same sequences as the 1^{st} to n^{th} positive control probes, excluding the k^{th} positive control probe, are tandemly joined to one another such that the end of at least one sequence overlaps with the end of other at least one sequence.

15. A method of detecting a plurality of nucleic acids, wherein 1^{st} to n^{th} (n: a natural number of 2 or more) nucleic acid samples are detected, comprising:
a first step of preparing a nucleic acid sample detection device including 1^{st} to n^{th} wells corresponding to the 1^{st} to n^{th} nucleic acid samples respectively, wherein the 1^{st} well includes a 1^{st} detection nucleic acid probe immobilization region on which a 1^{st} detection nucleic acid probe is immobilized for detecting the 1^{st} nucleic acid sample, a 1^{st} positive control immobilization region on which a 1^{st} positive control nucleic acid probe is immobilized for discriminating the 1^{st} nucleic acid sample, and a 1^{st} negative control immobilization region on which a 1^{st} negative control nucleic acid probe is immobilized for detecting contamination with a nucleic acid sample other than the 1^{st} nucleic acid sample, and the k^{th} (k: a natural number of 2 to n) well includes a k^{th} detection nucleic acid probe immobilization region on which a k^{th} detection nucleic acid probe is immobilized for detecting the k^{th} nucleic acid sample, a k^{th} positive control immobilization region on which a k^{th} positive control nucleic acid probe is immobilized for discriminating the k^{th} nucleic acid sample, and a k^{th} negative control immobilization region on which a k^{th} negative control nucleic acid probe is immobilized for detecting contamination with a nucleic acid sample other than the k^{th} nucleic acid sample;
a second step of preparing 1^{st} to n^{th} nucleic acid sample discrimination reagents, wherein the 1^{st} nucleic acid sample discrimination reagent is composed of a 1^{st} positive control judgment reagent containing a nucleic acid having a sequence complementary to the 1^{st} positive control nucleic acid probe immobilized on the 1^{st} positive control immobilization region and a 1^{st} negative control judgment reagent containing a plurality of nucleic acid having sequences complementary respectively to 2^{nd} to n^{th} negative control nucleic acid probes immobilized on 2^{nd} to n^{th} negative control immobilization regions, and the k^{th} (k: a natural number of 2 to n) nucleic acid sample discrimination reagent is composed of a k^{th} positive control judgment reagent containing a nucleic acid having a sequence complementary to the k^{th} positive control nucleic acid probe immobilized on the k^{th} positive control immobilization region and a k^{th} negative control judgment reagent containing a plurality of nucleic acid having sequences complementary respectively to the 1^{st} to n^{th} negative control nucleic acid probes, excluding the k^{th} negative control nucleic acid probe, immobilized on the 1^{st} to n^{th} negative control immobilization regions, excluding the k^{th} negative control immobilization region;
a third step of adding the 1^{st} to n^{th} nucleic acid sample discrimination reagents to the 1^{st} to n^{th} nucleic acid samples respectively;
a fourth step of injecting the 1^{st} to n^{th} nucleic acid samples into the 1^{st} to n^{th} wells respectively;
a fifth step of detecting the presence or absence of a reaction in the 1^{st} to n^{th} positive control immobilization regions;
a sixth step of detecting the presence or absence of a reaction in the 1^{st} to n^{th} negative control immobilization regions; and
a seventh step of detecting the presence or absence of a reaction in 1^{st} to n^{th} detection nucleic acid probe immobilization regions.

16. The method according to claim 15, wherein each of the 1^{st} to n^{th} wells is constructed as an independent nucleic acid sample detection device.

17. The method according to claim 15, wherein the 1st to n^{th} wells are arranged on 1 nucleic acid sample detection device.

18. A kit for detecting a plurality of nucleic acid samples, comprising:
the nucleic acid sample detection device prepared in the method according to claim 15; and
the 1^{st} to n^{th} nucleic acid sample discrimination reagents prepared in the method according to claim 15.

19. The kit according to claim 18, wherein the 1^{st} to n^{th} nucleic acid sample discrimination reagents are composed of 1^{st} to n^{th} positive control judgment reagents and 1^{st} to n^{th} negative control judgment reagents.

20. A method of detecting a plurality of nucleic acid samples, wherein 1^{st} to n^{th} (n: a natural number of 2 or more) nucleic acid samples are detected, comprising:
a first step of preparing a nucleic acid sample detection device including 1^{st} to n^{th} wells corresponding to the 1^{st} to n^{th} nucleic acid samples respectively, wherein the 1^{st} well includes a 1^{st} detection nucleic acid probe immobilization region on which a 1^{st} detection nucleic acid probe is immobilized for detecting the 1^{st} nucleic acid sample, a 1^{st} positive control immobilization region on which a 1^{st} positive control nucleic acid probe is immobilized for discriminating the 1^{st} nucleic acid sample, and a 1^{st} negative control immobilization region on which a n^{th} negative control nucleic acid probe is immobilized for detecting contamination with a nucleic acid sample other than the 1^{st} nucleic acid sample, and the k^{th} (k: a natural number of 2 to n) well includes a k^{th} detection nucleic acid probe immobilization region on which a k^{th} detection nucleic acid probe is immobilized for detecting the k^{th} nucleic acid sample, a k^{th} positive control immobilization region on which a k^{th} positive control nucleic acid probe is immobilized for discriminating the k^{th} nucleic acid sample, and a k^{th} negative control immobilization region on which a k^{th} negative control nucleic acid probe is immobilized for detecting contamination with a nucleic acid sample other than the k^{th} nucleic acid sample;
a second step of preparing 1^{st} to n^{th} nucleic acid sample discrimination reagents, wherein,
the 1^{st} nucleic acid sample discrimination reagent is composed of a 1^{st} positive control judgment reagent containing a nucleic acid which have sequence complementary to the 1^{st} positive control nucleic acid probe immobilized on the 1^{st} positive control immobilization region and a 1^{st} negative control judgment reagent containing a plurality of nucleic acids which have sequences complementary respectively to 2^{nd} to n^{th} negative control nucleic acid probes immobilized on 2^{nd} to n^{th} negative control immobilization regions and which have sequences complementary respectively to nucleic acids hybridizing with the 2^{nd} to n^{th} positive control nucleic acid probes immobilized on the 2^{nd} to n^{th} positive control immobilization regions, and
the k^{th} (k: a natural number of 2 to n) nucleic acid sample discrimination reagent is composed of a k^{th} positive control judgment reagent containing a nucleic acid having a sequence complementary to the k^{th} positive control nucleic acid probe immobilized on the k^{th} positive control immobilization region and a k^{th} negative control judgment reagent containing a plurality of nucleic acids which have sequences complementary respectively to the 1^{st} to n^{th} negative control nucleic acid probes, excluding the k^{th} negative control nucleic acid probe, immobilized on the 1^{st} to n^{th} negative control immobilization regions, excluding the k^{th} negative control immobilization region, and which have sequences complementary respectively to nucleic acids hybridizing with the 1^{st} to n^{th} positive control nucleic acid probes, excluding the k^{th} positive control nucleic acid probe, immobilized on the 1^{st} to n^{th} positive control immobilization regions, excluding the k^{th} positive control immobilization region;
a third step of adding the 1^{st} to n^{th} nucleic acid sample discrimination reagents to the 1^{st} to n^{th} nucleic acid samples respectively;
a fourth step of injecting the 1^{st} to n^{th} nucleic acid samples into the 1^{st} to n^{th} wells respectively;
a fifth step of detecting the presence or absence of a reaction in the 1^{st} to n^{th} positive control immobilization regions;
a sixth step of detecting the presence or absence of a reaction in the 1^{st} to n^{th} negative control immobilization regions; and
a seventh step of detecting the presence or absence of a reaction in 1^{st} to n^{th} detection nucleic acid probe immobilization regions.

21. The method according to claim 20, wherein the 1^{st} negative control judgment reagent comprises a plurality of nucleic acids wherein nucleic acids having sequences complementary respectively to the 2^{nd} to n^{th} negative control nucleic acid probes immobilized on the 2^{nd} to n^{th} negative control immobilization regions, and nucleic acids having sequences complementary respectively to nucleic acids hybridizing with the 2^{nd} to n^{th} positive control nucleic acid probes immobilized on the 2^{nd} to n^{th} positive control immobilization regions, are tandemly joined to one another, and
the k^{th} (k: a natural number of 2 to n) negative control judgment reagent comprises a plurality of nucleic acids wherein nucleic acids having sequences complementary respectively to the 1^{st} to n^{th} negative control nucleic acid probes, excluding the k^{th} negative control nucleic acid probe, immobilized on the 1^{st} to n^{th} negative control immobilization regions, excluding the k^{th} negative control immobilization region, and nucleic acids having sequences complementary respectively to nucleic acids hybridizing with the 1^{st} to n^{th} positive control nucleic acid probes excluding the k^{th} positive control nucleic acid probe, are tandemly joined to one another.

22. The method according to claim 20, wherein the 1^{st} negative control judgment reagent comprises a plurality of nucleic acids wherein nucleic acids having sequences complementary respectively to the 2^{nd} to n^{th} negative control nucleic acid probes immobilized on the 2^{nd} to n^{th} negative control immobilization regions, and nucleic acids having sequences complementary respectively to nucleic acids hybridizing with the 2^{nd} to n^{th} positive control nucleic acid probes immobilized on the 2^{nd} to n^{th} positive control immobilization regions, are tandemly joined to one another such that the end of each nucleic acid overlaps with the end of another nucleic acid, and
the k^{th} (k: a natural number of 2 to n) negative control judgment reagent comprises a plurality of nucleic acids wherein nucleic acids having sequences complementary respectively to the 1^{st} to n^{th} negative control nucleic acid probes, excluding the k^{th} negative control nucleic acid probe, immobilized on the 1^{st} to n^{th} negative control immobilization regions, excluding the k^{th} negative control immobilization region, and nucleic acids having sequences complementary respectively to nucleic acids hybridizing with the 1^{st} to n^{th} positive control nucleic acid probes excluding the k^{th} positive control nucleic acid probe, are tandemly joined to one another such that the end of each nucleic acid overlaps with the end of another nucleic acid.

23. The method according to claim 20, wherein each of the 1^{st} to n^{th} wells is constructed as an independent nucleic acid sample detection device.

24. The method according to claim 20, wherein the 1^{st} to n^{th} wells are arranged on 1 nucleic acid sample detection device.

25. A kit for detecting a plurality of nucleic acid samples, comprising:
the nucleic acid sample detection device prepared in the method according to claim 20, and
the 1^{st} to n^{th} nucleic acid sample discrimination reagents prepared in the method according to claim 20.

26. The kit according to claim 25, wherein the 1^{st} to n^{th} nucleic acid sample discrimination reagents are composed of 1^{st} to n^{th} positive control judgment reagents and 1^{st} to n^{th} negative control judgment reagents.


SEQUENCE LISTING (20 sequences)

SEQ ID NO: 1 - DNA, 18 bp, Artificial Sequence
  Synthetic DNA; Probe
ttcagttatgtggatgat

SEQ ID NO: 2 - DNA, 18 bp, Artificial Sequence
  Synthetic DNA; Probe
tcagttatgtcgatgatg

SEQ ID NO: 3 - DNA, 21 bp, Artificial Sequence
  Synthetic DNA; Probe
tttcagttatgttgatgatgt

SEQ ID NO: 4 - DNA, 20 bp, Artificial Sequence
  Synthetic DNA; Probe
tttcagttatgtagatgatg

SEQ ID NO: 5 - DNA, 15 bp, Artificial Sequence
  Synthetic DNA; Probe
tccgggcgcagaaac

SEQ ID NO: 6 - DNA, 14 bp, Artificial Sequence
  Synthetic DNA; Probe
gtgctgcaggtgcg

SEQ ID NO: 7 - DNA, 15 bp, Artificial Sequence
  Synthetic DNA; Probe
cgtgatgacaccaag

SEQ ID NO: 8 - DNA, 15 bp, Artificial Sequence
  Synthetic DNA; Probe
atgctttccgtggca

SEQ ID NO: 9 - DNA, 34 bp, Artificial Sequence
  Synthetic DNA; Probe
accaataaggtttattgaatatttgggcatcaga

SEQ ID NO: 10 - DNA, 30 bp, Artificial Sequence
  Synthetic DNA; Probe
tgcttctacacagtctcctgtacctgggca

SEQ ID NO: 11 - DNA, 30 bp, Artificial Sequence
  Synthetic DNA; Probe
tggtcctggcactgataatagggaatgtat

SEQ ID NO: 12 - DNA, 30 bp, Artificial Sequence
  Synthetic DNA; Probe
agtagttatgtatatgccccctcgcctagt

SEQ ID NO: 13 - DNA, 18 bp, Artificial Sequence
  Synthetic DNA; Reagent
atcatccacataactgaa

SEQ ID NO: 14 - DNA, 18 bp, Artificial Sequence
  Synthetic DNA; Reagent
catcatcgacataactga

SEQ ID NO: 15 - DNA, 21 bp, Artificial Sequence
  Synthetic DNA; Reagent
acatcatcaacataactgaaa

SEQ ID NO: 16 - DNA, 20 bp, Artificial Sequence
  Reagent
catcatctacataactgaaa

SEQ ID NO: 17 - DNA, 15 bp, Artificial Sequence
  Synthetic DNA;  Reagent
gtttctgcgcccgga

SEQ ID NO: 18 - DNA, 14 bp, Artificial Sequence
  Synthetic DNA; Reagent
cgcacctgcagcac

SEQ ID NO: 19 - DNA, 15 bp, Artificial Sequence
  Synthetic DNA; Reagent
cttggtgtcatcacg

SEQ ID NO: 20 - DNA, 15 bp, Artificial Sequence
  Synthetic DNA; Reagent
tgccacggaaagcat